(19) United States
(12) Patent Application Publication
(10) Pub. No.: US 2010/0172916 A1
(43) Pub. Date: Jul. 8, 2010

(54) SUBSTITUTED HYDROXYPHENYLAMINE COMPOUNDS

(75) Inventors: Thomas G. Gant, Carlsbad, CA (US); Craig Hodulik, San Diego, CA (US); Soon Woo, Vista, CA (US)

Correspondence Address: GLOBAL PATENT GROUP - APX, 10411 Clayton Road, Suite 304, ST. LOUIS, MO, 63131, US

(73) Assignee: AUSPEX PHARMACEUTICALS, INC., Vista, CA (US)

(21) Appl. No.: 12/614,530

(22) Filed: Nov. 9, 2009

Related U.S. Application Data
(60) Provisional application No. 61/112,788, filed on Nov. 10, 2008.

(51) Int. Cl. A61K 31/7084; A61K 31/122; A61K 38/44; A61K 33/26; A61K 36/48; A61K 31/714; A61K 31/675; A61K 31/66; A61K 31/55; A61K 31/551; A61K 31/5375; A61K 31/519; A61K 31/4985; A61K 31/525; A61K 31/506; A61K 31/515; A61K 31/435; A61K 31/473; A61K 31/48; A61K 31/437; A61K 31/4545; A61K 31/4415; A61K 31/455; A61K 31/4409; A61K 31/428; A61K 31/421; A61K 31/4045; A61K 31/381; A61K 31/355; A61K 31/375; A61K 31/197; A61K 31/165; A61K 31/135; C07C 229/36; C07C 211/03; A61P 25/00; A61P 3/00; A61P 3/04; A61P 25/16; A61P 25/24; A61P 25/28; A61P 25/22; A61P 25/30; A61P 31/18; A61K 39/395

(52) U.S. Cl. 424/158.1; 424/94.1; 424/94.4; 424/648; 424/757; 514/47; 514/52; 514/89; 514/129; 514/217.02; 514/221; 514/237.8; 514/249; 514/250; 514/251; 514/252.16; 514/252.19; 514/270; 514/284; 514/288; 514/289; 514/293; 514/318; 514/348; 514/355; 514/357; 514/367; 514/376; 514/418; 514/438; 514/458; 514/474; 514/567; 514/620; 514/655; 514/689; 560/40; 564/280

(57) ABSTRACT

The present invention relates to new substituted hydroxyphenylamine based modulators of hormone and/or pigment levels, pharmaceutical compositions thereof, and methods of use thereof.

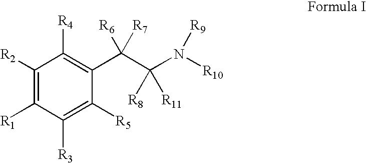

This application claims the benefit of priority of U.S. provisional application No. 61/112,788, filed Nov. 10, 2008, the disclosure of which is hereby incorporated by reference as if written herein in its entirety.

### FIELD

Disclosed herein are new substituted hydroxyphenylamine compounds, pharmaceutical compositions made thereof, and methods to modulate hormone, and/or pigment levels with such compounds for the treatment of disorders in a subject, such as stress-associated conditions, obesity, alcohol withdrawal syndrome, drug dependence, depression, Parkinson's disease, narcolepsy, Alzheimer's disease, phenylketonuria, multi-infarct dementia, vitiglio, chronic uremia, HIV infection of the central nervous system, AIDS dementia, amyotrophic lateral sclerosis, hereditary hemorrhage with amyloidosis-Dutch type, cerebral amyloid angiopathy, Down's syndrome, spongiform encephalopathy, Creutzfeldt-Jakob disease, hemorrhagic shock, restless leg syndrome, dystonia, carbon monoxide poisoning, cyanide poisoning, methanol poisoning, manganese poisoning, disorders associated with hormone levels, and/or disorders associated with pigment levels.

### BACKGROUND

Tyrosine or 4-hydroxyphenylalanine is one of the twenty common amino acids found in nature. Tyrosine is a nonessential amino acid in humans, and is synthesized from phenylalanine. Tyrosine is used by cells for protein biosynthesis, and plays a critical role in many signal transduction pathways. Additionally, tyrosine is the precursor for many neurotransmitters, hormones, and pigments. Tyrosine supplementation was found to be beneficial during conditions of stress, cold, fatigue, (Hao et al., Pharmacol. Biochem. Behav. 2001, 68(2), 273-81), prolonged work and sleep deprivation (Magill et al., Nutritional Neuroscience 2003, 6(4), 237-46; and Neri et al., Aviation, space, and environmental medicine 1995, 66 (4), 313-9), conditions with reductions in stress hormone levels (Reinstein et al., Life Sci. 1985, 37(23), 2157-63), obesity (Hao et al., Pharmacol. Biochem. Behav. 2001, 68(2), 273-81), and improvements in cognitive and physical performance (Thomas et al., Pharmacol Biochem Behav. 1999, 64(3), 495-500; Deijen et al., Brain Res. Bull. 1994, 3, 319-23; and Mahoney et al., Physiol and Behav. 2007, 92(4), 575-82). Additionally, tyrosine (m-tyrosine and/or p-tyrosine) and/or tyramine (m-tyramine and/or p-tyramine) supplementation was beneficial in treating or likely beneficial in treating the following disorders: alcohol withdrawal syndrome (Blum K, Integr Psychiatr 1986, 6, 199-204), drug dependence (Blum K, Integr Psychiatr 1986, 6, 199-204; and Geis et al., Pharmacol Biochem Behav. 1986, 25(5), 1027-33), depression (Goldberg I K, Lancet 1980, 2, 364; Gelenberg et al. Am J Psychiatry 1980, 137, 622-3), Parkinson's disease (Lemoine et al., Comples Rendus Academie des sciences (III) 1989, 309(2), 43-47; Ungerstedt et al., European J of Pharmacology 1973, 21, 230-237; Yamaguchi T et al., Science 1983, 219(4580), 75-7; and Young S., Neurosci Biobehav Rev. 1996, 20(2), 313-23), narcolepsy (Elwes et al., Lancet 1989, 2(8671), 1067-9), Alzheimer's disease (Meyer et al., J Am Ger Soc 1977, 25(7), 289-298; U.S. Pat. No. 6,043,283 A), phenylketonuria (PKU) (Koch R. Am J Clin Nutr 1996, 64, 974-5), multi-infarct dementia (Meyer et al., J Am Ger Soc 1977, 25(7), 289-298), phenylketonuria (PKU) (Koch R. Am J Clin Nutr 1996, 64, 974-5), chronic uremia (Alvestrand et al., Clin Nephrol 1983, 19, 67-73), vitiglio (Antoniou et al., Int J Dermatol 1989, 28(8), 545-7; and Anderson et al., J Nutr. 2002, 132(7), 2037-42), HIV infection of the central nervous system (U.S. Pat. No. 6,043,283 A), AIDS dementia (U.S. Pat. No. 6,043,283 A), amyotrophic lateral sclerosis (U.S. Pat. No. 6,043,283 A), hereditary hemorrhage with amyloidosis-Dutch type (U.S. Pat. No. 6,043,283 A), cerebral amyloid angiopathy (U.S. Pat. No. 6,043,283 A), Down's syndrome (U.S. Pat. No. 6,043,283 A), spongiform encephalopathy (U.S. Pat. No. 6,043,283 A), Creutzfeldt-Jakob disease (U.S. Pat. No. 6,043,283 A), hemorrhagic shock, (Simon et al., Arch Sure 1987, 122 (1), 78), and dystonia (Morton et al., Pediatrics 2002, 109(6), 999-1008).

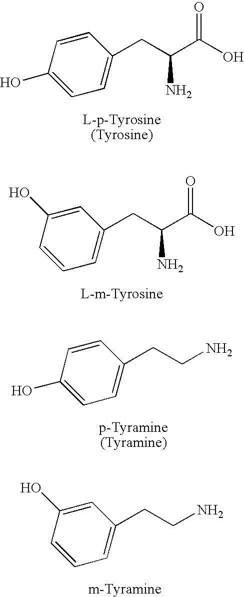

Three isomers of tyrosine are known. In addition to the common amino acid L-tyrosine, the para isomer (para-tyr, p-tyr, or 4-hydroxyphenylalanine), there are two additional regioisomers, namely meta-tyrosine (m-tyr, 3-hydroxyphenylalanine, or L-m-tyrosine) and ortho-tyrosine (o-tyr, or 2-hydroxyphenylalanine). The m-tyr and o-tyr isomers, which are rare in humans, arise through non-enzymatic free-radical hydroxylation of phenylalanine under conditions of oxidative stress. Tyrosine is metabolized by various enzymatic pathways. Whether tyrosine is first transaminated, de-carboxylated, or hydroxylated, determines the metabolic fate of tyrosine.

Tyrosine can be hydroxylated to give levodopa (L-dopa) in the adrenal gland by tyrosine hydroxylase (TH). L-dopa is a very minor product of tyrosine metabolism. The vast majority of detectable tyrosine metabolites result from transamination- or de-carboxylation-based pathways. L-dopa is metabolized in the brain to dopamine by aromatic L-amino acid decarboxylase. Dopamine can be further processed into norepinephrine by dopamine beta-hydroxylase. Dopamine has many functions in the brain, including important roles in behavior and cognition, motor activity, motivation and reward, inhibition of prolactin production (involved in lactation), sleep, mood, attention, and learning. Since L-dopa is derived from tyrosine (including m-tyr), tyrosine supplementation may increase depressed neurotransmitter levels, such as dopamine (Young S., Neurosci Biobehav Rev. 1996, 20(2), 313-23; and Montgomery A., Am J Psychiatry 2003, 160(10), 1887-9).

Tyrosine (p-tyr, m-tyr, and o-tyr) is de-carboxylated to tyramine (p-tyramine, m-tyramine, or o-tyramine) by monoamine oxidases (MAOs). Tyramine (p-tyramine and m-tyramine) can cause the release of stored monoamines, such as dopamine, norepinephrine, and epinephrine, and can also act directly as a neurotransmitter to affect blood pressure. An increased tyramine level may therefore be beneficial to subjects suffering from disorders resulting from depressed levels of neurotransmitters in dopaminergic neurons, such as Parkinson's disease (Ungerstedt et al., European J of Pharmacology 1973, 21, 230-237). A large dietary intake of tyramine (or a dietary intake of tyramine while taking MAO inhibitors) can cause the ‘tyramine pressor response,’ which is defined as an increase in systolic blood pressure of 30 mmHg or more. With repeated exposure to high levels of tyramine, however, there is a decreased pressor response; tyramine is degraded to octopamine, which is subsequently packaged in synaptic vesicles with norepinephrine (noradrenaline). Therefore, after repeated tyramine exposure, these vesicles contain an increased amount of octopamine and a relatively reduced amount of norepinephrine.

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Tyrosine and/or tyramine are substituted hydroxyphenylamine-based modulators of hormone, and/or pigment levels. The carbon-hydrogen bonds of tyrosine and tyramine contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Kinetic Isotope Effect (KIE) that could affect the pharmacokinetic, pharmacologic and/or toxicologic profiles of tyrosine and/or tyramine in a subject in comparison with tyrosine and/or tyramine having naturally occurring levels of deuterium.

Based on discoveries made in our laboratory, as well as considering the KIE literature, tyrosine is metabolized by various enzymatic pathways, including: decarboxylation to form tyramine; hydroxylation to form L-Dopa; and transamination to form hydroxyphenylpyruvate. Tyramine is oxidized by monoamine oxidase to form octopamine. The current approach has the potential to prevent or retard metabolism at these sites, such as retarding the conversion of tyramine to octopamine, or alternatively shunting metabolism to a more favored enzymatic pathway, such as hydroxylation of tyrosine to L-Dopa. Other sites on the molecule may also undergo transformations leading to metabolites with as-yet-unknown pharmacology/toxicology. Limiting the production of such metabolites has the potential to decrease the danger of the administration of such drugs and may even allow increased dosage and concomitant increased efficacy. All of these transformations, among other potential transformations, can occur through polymorphically-expressed enzymes, leading to interpatient variability. Further, it is quite typical for disorders ameliorated by the present invention, such as Parkinson's disease, to produce symptoms that are best medicated around the clock for extended periods of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the potential to slow the metabolism and/or selectively shunt the metabolism of tyrosine and/or tyramine to more favorable enzymatic pathways.

Novel compounds and pharmaceutical compositions, certain of which have been found to modulate hormone and/or pigment levels have been discovered, together with methods of synthesizing and using the compounds, including methods for the treatment of hormone-mediated disorders and/or pigment-mediated disorders in a patient by administering the compounds as disclosed herein.

In certain embodiments of the present invention, compounds have structural Formula I:

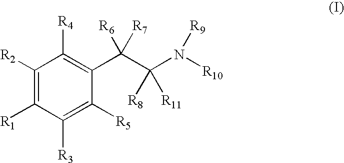

or a pharmaceutically acceptable salt, solvate, or prodrug thereof, wherein:

R₁ and R₂ are independently selected from the group consisting of hydrogen, deuterium, —OH, and —OD, wherein at least one of R₁ or R₂ is hydrogen or deuterium;

R₃-R₁₀ are independently selected from the group consisting of hydrogen and deuterium;

R₁₁ is selected from the group consisting of hydrogen, deuterium, CO₂H, —CO₂D, and —CO₂R₁₂, wherein R₁₂ is an alkyl, or deuterated alkyl; and

at least one of R₁-R₁₂ is deuterium or contains deuterium.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In other embodiments the compound cannot be selected from the group consisting of:

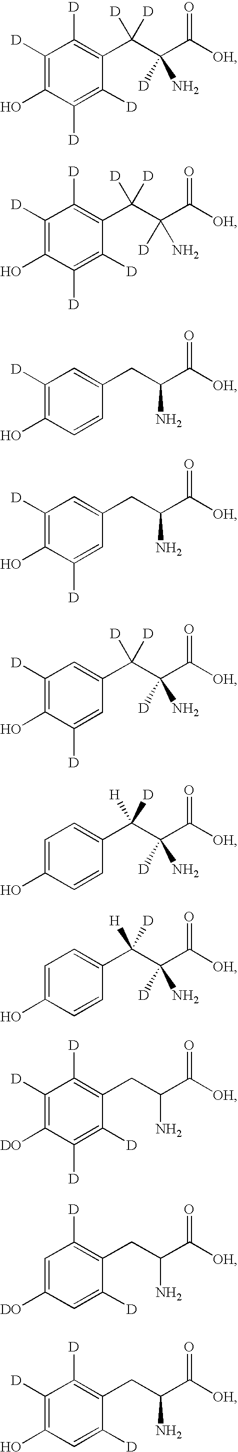

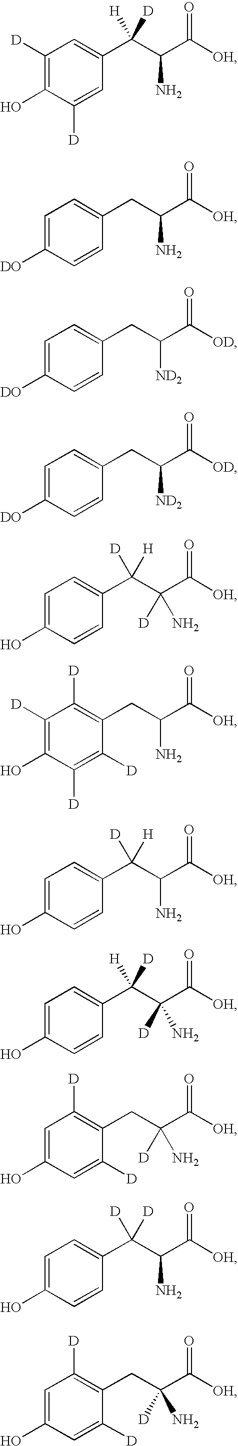

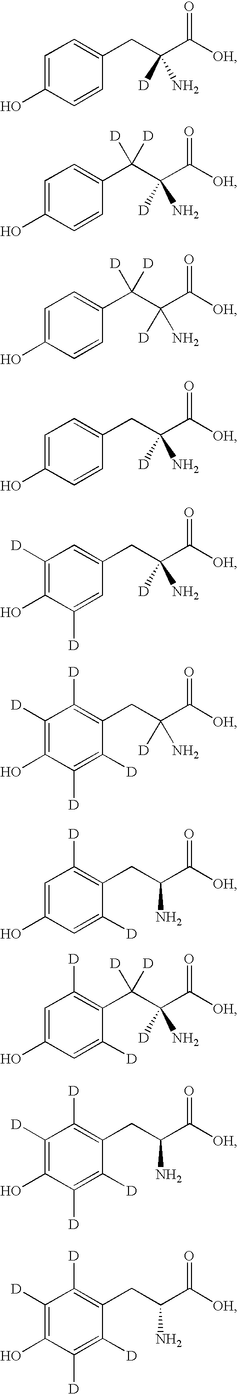

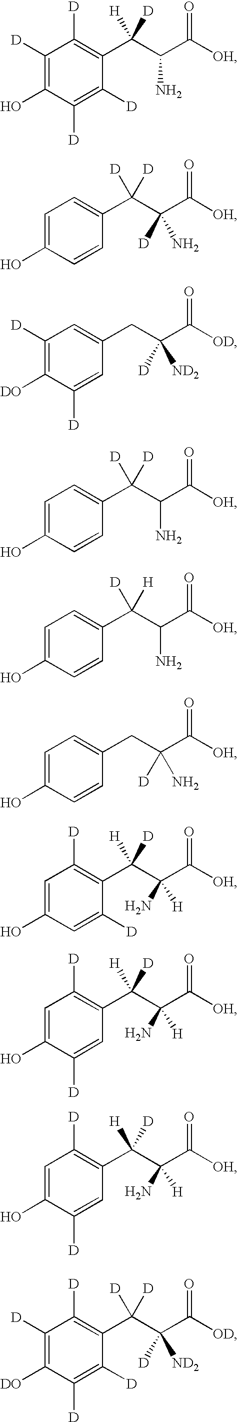

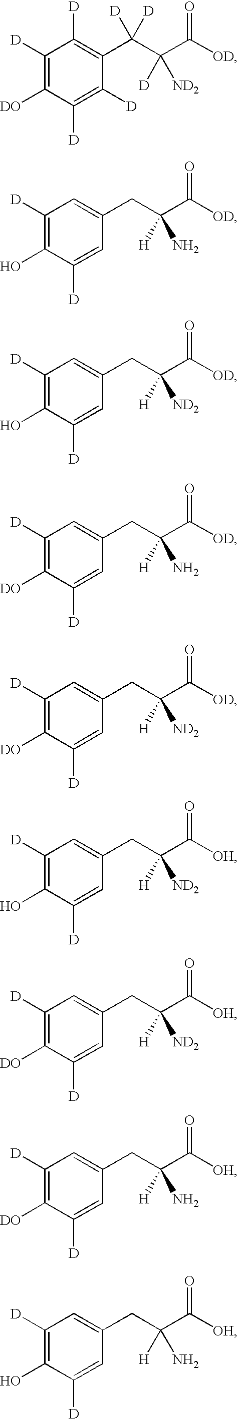

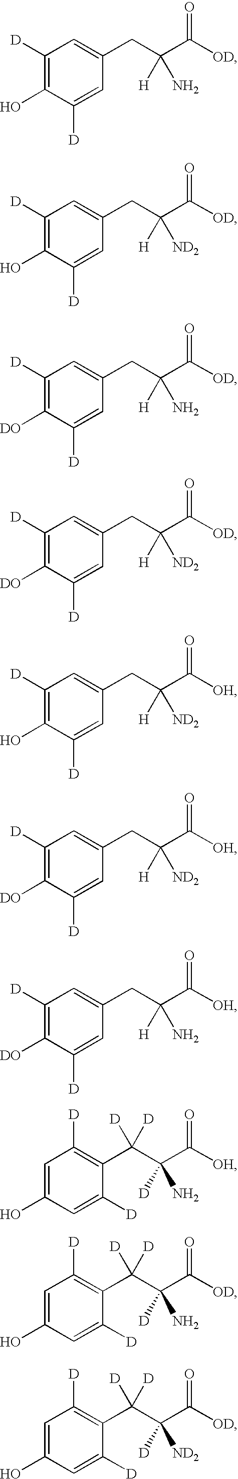

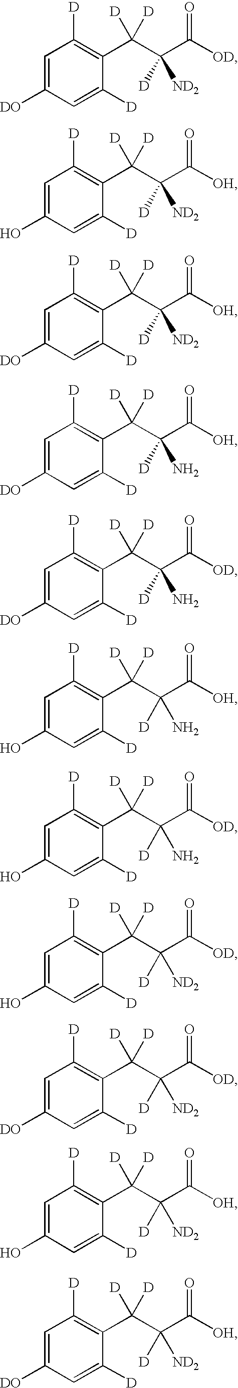

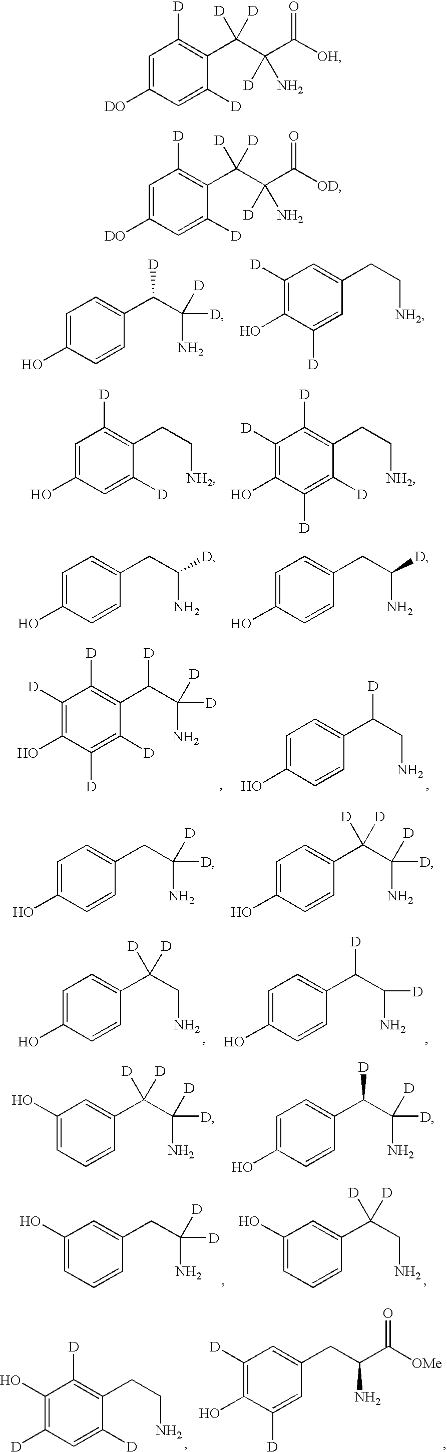

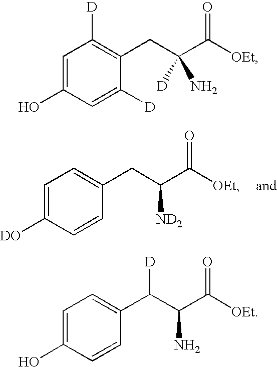

Certain compounds disclosed herein may be useful in modulating hormone and/or pigment levels, and may be used in the treatment or prophylaxis of a disorder in which hormone, and/or pigment levels play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating hormonal and/or pigment levels. Other embodiments provide methods for treating a hormone-mediated disorder and/or a pigment-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disorder ameliorated by administering a modulator of hormone and/or pigment levels.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cmax) of the minimum efficacious dose (MED), lowering the efficacious dose and thus decreasing the non-mechanism-related toxicity, and/or lowering the probability of drug-drug interactions.

In another aspect are processes for preparing a compound as disclosed herein or other pharmaceutically acceptable derivative thereof such as a salt, solvate, or prodrug, as a modulator of hormone, and/or pigment levels.

In other embodiments, at least at least one of R₁-R₁₂ has deuterium enrichment of no less than about 10%, 50%, 90%, or 98%.

In other embodiments, a pharmaceutical composition comprises a compound disclosed herein together with a pharmaceutically acceptable carrier.

In certain embodiments of the present invention a method of treating a subject suffering from a hormone-mediated disorder and/or pigment-mediated disorder comprises the administration of a therapeutically effective amount of a compound as disclosed herein.

In other embodiments said hormone-mediated disorder and/or pigment-mediated disorder is selected from the group consisting of stress-associated conditions, obesity, alcohol withdrawal syndrome, drug dependence, depression, Parkinson's disease, narcolepsy, Alzheimer's disease, phenylketonuria, multi-infarct dementia, vitiglio, chronic uremia, HIV infection of the central nervous system, AIDS dementia, amyotrophic lateral sclerosis, hereditary hemorrhage with amyloidosis-Dutch type, cerebral amyloid angiopathy, Down's syndrome, spongiform encephalopathy, Creutzfeldt-Jakob disease, hemorrhagic shock, restless leg syndrome, dystonia, carbon monoxide poisoning, cyanide poisoning, methanol poisoning, or manganese poisoning, any disorder associated with abnormal hormone levels, and/or any disorder associated with abnormal pigment levels.

In yet other embodiments, said method further comprises the administration of an additional therapeutic agent.

In further embodiments said therapeutic agent is selected from the group consisting of: dietary supplements, dopamine agonists, monoamine oxidase inhibitors, dopamine prodrugs, L-dopa metabolism suppressors, adamantine-based agents, SNRIs, SSRIs, acetylcholinesterase inhibitors, TCAs, barbituates, benzodiazepines, amphetamine-like stimulants, platelet aggregation inhibitors, statins, anticoagulants, thrombolytics, fibrates, bile acid sequestrants, CETP inhibitors, lipid modifying agents, NSAIDs, anti-bacterial agents, anti-fungal agents, sepsis treatments, steroidals, local or general anesthetics, NRIs, DARIs, sedatives, NDRIs, SNDRIs, monoamine oxidase inhibitors, hypothalamic phospholipids, ECE inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIIa Inhibitors and Factor Xa Inhibitors, renin inhibitors, NEP inhibitors, vasopeptidase inhibitors, squalene synthetase inhibitors, anti-atherosclerotic agents, MTP Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, antiinflammatories, antiproliferatives, chemotherapeutic agents, immunosuppressants, anticancer agents and cytotoxic agents, antimetabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

In certain embodiments, the compounds provided herein can be combined with one or more dietary supplements known in the art, including, but not limited to, ferrous iron, tetrahydrofolic acid, pyridoxal phosphate, NADH, pyridoxine, nicotinamide, vitamin C, vitamin E, vitamin B12, vitamin B3, curcumin, folic acid, Coenzyme Q10, Mucuna pruriens extract, and MitoQ.

In certain embodiments, the compounds disclosed herein can be combined with one or more dopamine agonists known in the art, including, but not limited to, A-412,997, apomorphine, bromocriptine, cabergoline, dihydrexidine, dihydroergocryptine mesylate, fenoldopam, lisuride, pergolide, piribedil, pramipexole, propylnorapomorphine, quinpirole, ropinirole, rotigotine, SKF 38393, and SKF 82958.

In certain embodiments, the compounds disclosed herein can be combined with one or more monoamine oxidase inhibitors known in the art, including, but not limited to, iproclozide, iproniazid, isocarboxazid, nialamide, pargyline, phenelzine, rasagiline, selegiline, toloxatone, tranylcypromine, brofaromine, beta-carbolines (harmaline) and moclobemide, linezolid, and dienolide kavapyrone desmethoxyyangonin.

In certain embodiments, the compounds disclosed herein can be combined with one or more dopamine prodrugs known in the art, including, but not limited to droxidopa, levodopa, melevodopa, and etilevodopa.

In certain embodiments, the compounds provided herein can be combined with one or more L-dopa metabolism suppressors known in the art, including, but not limited to, carbidopa, benserazide, tolcapone, and entacapone.

In certain embodiments, the compounds provided herein can be combined with adamantine-based agents known in the art, including, but not limited to, amantadine, memantine, and rimantadine.

In certain embodiments, the compounds disclosed herein can be combined with one or more SNRIs known in the art, including, but not limited to bicifadine, desvenlafaxine, duloxetine, milnacipran, nefazodone, and venlafaxine.

In certain embodiments, the compounds disclosed herein can be combined with one or more SSRIs known in the art, including, but not limited to alaproclate, citalopram, dapoxetine, escitalopram, etoperidone, fluoxetine, fluvoxamine, paroxetine, sertraline, and zimelidine.

In certain embodiments, the compounds disclosed herein can be combined with one or more acetylcholinesterase inhibitors known in the art, including, but not limited to metrifonate, physostigmine, neostigmine, pyridostigmine, ambenonium, demarcarium, rivastigmine, galantamine, donepezil, tacrine, and edrophonium.

In certain embodiments, the compounds disclosed herein can be combined with one or more TCAs known in the art, including, but not limited to clomipramine, nefazodone, trazodone, amitriptyline, amoxapine, butriptyline, desipramine/lofepramine, dibenzepin, dothiepin, doxepin, imipramine, iprindole, melitracen, nortriptyline, opipramol, protriptyline, trimipramine, maprotiline and amineptine.

In certain embodiments, the compounds provided herein can be combined with one or more barbituates known in the art, including, but not limited to, allobarbital, alphenal, amobarbital, aprobarbital, barbexaclone, barbital, brallobarbital, brophebarbital, bucolome, butabarbital, butalbital, butobarbital, butallylonal, crotylbarbital, cyclobarbital, cyclopal, enallylpropymal, ethallobarbital, febarbamate, heptabarbital, hexethal, hexobarbital, mephobarbital, metharbital, methohexital, methylphenobarbital, narcobarbital, nealbarbital, pentobarbital, phenobarbital, phetharbital, prazitone, probarbital, propallylonal, proxibarbal, roxibarbital, reposal, secbutabarbital, secobarbital, sigmodal, spirobarbital, talbutal, thialbarbital, thiamylal, thiobarbital, thiobutabarbital, thiopental, valofane, vinbarbital, and vinylbital.

In certain embodiments, the compounds disclosed herein can be combined with one or more benzodiazepines (“minor tranquilizers”) known in the art, including, but not limited to alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

In certain embodiments, the compounds disclosed herein can be combined with one or more amphetamine-like stimulants known in the art, including, but not limited to the group including 4-bromomethcathinone, 4-fluoroamphetamine, 4-fluoromethamphetamine, 4-fluoromethcathinone, 4-methylmethcathinone, aletamine, amfepentorex, amphechloral, racemic amphetamine salts (dextroamphetamine, Adderall), amphetaminil, benzphetamine, bupropion, cathinone, chlorphentermine, clenbuterol, clobenzorex, clortermine, diethylpropion, dimethoxyamphetamine, dimethylamphetamine, dimethylcathinone, ephedrine, epinephrine, ethcathinone, ethylamphetamine, fenethylline, fenfluramine, fenproporex, fludorex, furfenorex, levomethamphetamine, misdexamfetamine, MDMA, mefenorex, methamphetamine, methcathinone, methoxyphedrine, methylone, octopamine, ortetamine, parahydroxyamphetamine, PCA, PIA, PMA, PMEA, PMMA, PPAP, phendimetrazine, phenmetrazine, phentermine, phenylephrine, phenylpropanolamine, propylamphetamine, pseudoephedrine, selegiline, synephrine, tiflorex, and xylopropamine.

In other embodiments said method further results in at least one effect selected from the group consisting of
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In other embodiments said method further results in at least two effects selected from the group consisting of:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In certain embodiments said method decreases metabolism by at least one polymorphically-expressed cytochrome P450 isoform in said subject per dosage unit thereof as compared to the non-isotopically enriched compound.

In other embodiments said cytochrome P450 isoform is selected from the group consisting of CYP2C8, CYP2C9, CYP2C19, and CYP2D6.

In yet further embodiments said method decreases inhibition of at least one cytochrome P450 or monoamine oxidase isoform in said subject per dosage unit thereof as compared to the non-isotopically enriched compound.

In certain embodiments said cytochrome P450 or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAOA, and MAOB.

In certain embodiments, said method reduces a deleterious change in a diagnostic hepatobiliary function endpoint, as compared to the corresponding non-isotopically enriched compound.

In yet other embodiments, said diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

In another embodiment a compound disclosed herein can be used as a medicament.

In a further embodiment a compound disclosed herein can be used in the manufacture of a medicament for the prevention or treatment of a disorder ameliorated by administering a modulator of hormone and/or pigment levels.

All publications and references cited herein are expressly incorporated herein by reference in their entirety. However, with respect to any similar or identical terms found in both the incorporated publications or references and those expressly put forth or defined in this document, then those terms definitions or meanings expressly put forth in this document shall control in all respects.

As used herein, the terms below have the meanings indicated.

The singular forms “a”, “an”, and “the” may refer to plural articles unless specifically stated otherwise.

The term “about”, as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

In representing a range of positions on a structure, the notation “from Rₓ . . . to Rₓₓ” or “Rₓ-Rₓₓ” may be used, wherein x and xx represent numbers. Then unless otherwise specified, this notation is intended to include not only the numbers represented by x and xx themselves, but all the numbered positions that are bounded by x and xx. For example, “from R₁ . . . to R₄” or “R₁-R₄” would, unless otherwise specified, be equivalent to R₁, R₂, R₃, and R₄.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₁₂ or the symbol “D,” when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S”, depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a linkage between two atoms, or two moieties when the atoms joined by the bond are considered to be part of larger substructure. A bond may be ionic, metallic, or covalent. If covalent, the bond can be either result from the sharing of one pair of electrons, a single bond; a sharing of 2 pairs of electrons, a double bond; a sharing of 3 pairs of electrons, or a triple bond; or sharing of more than 3 pairs of electrons. A dashed line between two atoms in a drawing of a molecule indicates that an additional bond may be present or absent at that position.

The term “disorder” as used herein is intended to be generally synonymous, and is used interchangeably with, the terms “disease”, “syndrome”, and “condition” (as in medical condition), in that all reflect an abnormal condition of the human or animal body or of one of its parts that impairs normal functioning, is typically manifested by distinguishing signs and symptoms.

The terms “treat”, “treating”, and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent”, “preventing”, and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “hormone” refers to a chemical substance produced in the body that controls and regulates the activity of certain cells or organs. Many hormones are secreted by specialized glands such as the thyroid gland. Hormones are essential for every activity of daily living, including the processes of digestion, metabolism, growth, reproduction, and mood control. Many hormones, such as the neurotransmitters, are active in more than one physical process. Examples of hormones covered by this invention include but are not limited to, the thyroid hormones, thyroxine (T₄) and triiodothyronine (T₃); and the catecholamines, dopamine, epinephrine, and norepinephrine. Unless stated otherwise, the term “hormone,” includes prohormones and catecholamine associated prodrugs, such as L-dopa.

The term “pigment” refers to material resulting in color in a subject, which is the result of selective color absorption. A pigment, such as melanin, can also function as a photoprotectant, by protecting cells from harmful UV-radiation.

The term “hormone-mediated disorder” refers to a disorder that is characterized by abnormal hormone levels or normal hormone levels that, when that hormone level is modulated, leads to the amelioration of other abnormal biological processes. Hormone-mediated disorders may be completely or partially mediated by modulating the level of hormones in a subject. In particular, a hormone-mediated disorder is one in which modulating the level of hormones in a subject results in some effect on the underlying disorder, e.g., administering a modulator of hormone levels results in some improvement in at least some of the subjects being treated.

The term “pigment-mediated disorder” refers to a disorder that is characterized by abnormal pigment levels or normal pigment levels that, when that pigment level is modulated, leads to the amelioration of other abnormal biological processes. Pigment-mediated disorders may be completely or partially mediated by modulating the level of pigments in a subject. In particular, a pigment-mediated disorder is one in which modulating the level of pigments in a subject results in some effect on the underlying disorder, e.g., administering a modulator of pigment levels results in some improvement in at least some of the subjects being treated.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient”, “active compound”, and “active substance” refer to a compound, which is administered, alone or in combination with one or more pharmaceutically acceptable excipients or carriers, to a subject for treating, preventing, or ameliorating one or more symptoms of a disorder.

The terms “drug”, “therapeutic agent”, and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function does not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The term “alkylating reagent” refers to any electrophillic reagent capable of transferring an unsubstituted or substituted alkyl group to a nucleophile and as such would be obvious to one of ordinary skill and knowledge in the art. Alkylating reagents include, but are not limited to, compounds having the structure R₁₀₀-LG, where R₁₀₀ is an alkyl group and LG is a leaving group. Specific examples of alkylating reagents include iodomethane, dimethyl sulfate, dimethyl carbonate, methyl toluenesulfonate, and methyl methanesulfonate.

The terms “alkyl” and “substituted alkyl” are interchangeable and include substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (—CD₃), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group consisting of hydrogen, deuterium, halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₁₀₁ and —C₀₋₁₀alkylCONR₁₀₂R₁₀₃ wherein R₁₀₁, R₁₀₂ and R₁₀₃ are independently selected from the group consisting of hydrogen, deuterium, alkyl, aryl, or R₃₂ and R₃₃ are taken together with the nitrogen to which they are attached forming a saturated cyclic or unsaturated cyclic system containing 3 to 8 carbon atoms with at least one substituent as defined herein.

The compounds disclosed herein can and do exist as therapeutically acceptable salts. The term “pharmaceutically acceptable salt”, as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. The most suitable route for administration depends on a variety of factors, including interpatient variation or disorder type, and therefore the invention is not limited to just one form of administration. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 100 mg to 15 g/day. Tablets or other forms of presentation provided in discrete units may conveniently contain an amount of one or more compounds which is effective at such dosage or as a multiple of the same, for instance, units containing 1 mg to 3000 mg, usually around 100 mg to 1000 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a hormone-mediated disorder and/or a pigment-mediated disorder comprising administering to a subject having or suspected of having such a disorder, a therapeutically effective amount of a compound as disclosed herein or a pharmaceutically acceptable salt, solvate, or prodrug thereof.

Hormone-mediated disorders and/or pigment-mediated disorders, include, but are not limited to, stress-associated conditions, obesity, alcohol withdrawal syndrome, drug dependence, depression, Parkinson's disease, narcolepsy, Alzheimer's disease, phenylketonuria, multi-infarct dementia, vitiglio, chronic uremia, HIV infection of the central nervous system, AIDS dementia, amyotrophic lateral sclerosis, hereditary hemorrhage with amyloidosis-Dutch type, cerebral amyloid angiopathy, Down's syndrome, spongiform encephalopathy, Creutzfeldt-Jakob disease, hemorrhagic shock, restless leg syndrome, dystonia, carbon monoxide poisoning, cyanide poisoning, methanol poisoning, or manganese poisoning, disorders associated with hormone levels, and/or disorders associated with pigment levels.

In certain embodiments, a method of treating a hormone-mediated disorder, and/or a pigment-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder; (7) prevention of recurrence, or delay of decline or appearance, of abnormal alimentary or hepatic parameters as the primary clinical benefit; or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al., Rapid Communications in Mass Spectrometry 2005, 19, 1943-1950; Shimamura et al., Journal of Chromatography 1986, 374(1), 17-26; Birgitta Sjöquist, Biomedical Spectrometry 1979, 6(9), 392-395; Heinecke J. W., Methods in Biological Oxidative Stress 2003, 93-100; Ishimitsu et al., Chemical & Pharmaceutical Bulletin 1982, 30(5), 1889-91; Li et al., Journal of Pharmaceutical and Biomedical Analysis 2000, 24(2), 325-333, and any references cited therein and any modifications made thereof.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al., British Journal of Clinical Pharmacology 2000, 49, 343-351. The inhibition of the MAO_{A} isoform is measured by the method of Weyler et al., J. Biol. Chem. 1985, 260, 13199-13207. The inhibition of the MAO_{B} isoform is measured by the method of Uebelhack et al., Pharmacopsychiatry, 1998, 31, 187-192.

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, statistically-significant improvement in Unified Parkinson's Disease Rating Scale, Hoehn and Yahr scale, Schwab and England Activities of Daily Living Scale, Beck Depression Inventory, Beck Anxiety Inventory, Beck Hopelessness Scale, executive functions, proprioception, hyposmia, anosmia, weight loss, International Restless Legs Syndrome Study Group Scale, episodic memory, semantic memory, implicit memory, inflammation, and pain indices; statistically-significant decrease in the occurrence of tremors, muscular hypertonicity, akinesia, bradykinesia, postural instability, gait and posture disturbances, aboulia, dementia, short term memory loss, somnolence, insomnia, disturbingly vivid dreams, REM Sleep Disorder, dizziness, fainting, pain, altered sexual function, long term memory loss, inability to perform activities of daily learning, oral and dental disease, pressure ulcers, malnutrition, infections, and swallowing difficulties; decreased mortality; reduction in need for hemodialysis, and/or diminution of toxicity including but not limited to, hepatotoxicity or other toxicity, or a decrease in aberrant liver enzyme levels as measured by standard laboratory protocols, as compared to the corresponding non-isotopically enriched compound when given under the same dosing protocol including the same number of doses per day and the same quantity of drug per dose.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of hormone-mediated disorders and/or pigment-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds provided herein can be combined with one or more dietary supplements known in the art, including, but not limited to, ferrous iron, tetrahydrofolic acid, pyridoxal phosphate, NADH, pyridoxine, nicotinamide, vitamin C, vitamin E, vitamin B12, vitamin B3, curcumin, folic acid, Coenzyme Q10, Mucuna pruriens extract, and MitoQ.

In certain embodiments, the compounds disclosed herein can be combined with one or more dopamine agonists known in the art, including, but not limited to, A-412,997, apomorphine, bromocriptine, cabergoline, dihydrexidine, dihydroergocryptine mesylate, fenoldopam, lisuride, pergolide, piribedil, pramipexole, propylnorapomorphine, quinpirole, ropinirole, rotigotine, SKF 38393, and SKF 82958.

In certain embodiments, the compounds disclosed herein can be combined with one or more monoamine oxidase inhibitors known in the art, including, but not limited to iproclozide, iproniazid, isocarboxazid, nialamide, pargyline, phenelzine, rasagiline, selegiline, toloxatone, tranylcypromine, brofaromine, beta-carbolines (harmaline) and moclobemide, linezolid, and dienolide kavapyrone desmethoxyyangonin.

In certain embodiments, the compounds disclosed herein can be combined with one or more dopamine prodrugs known in the art, including, but not limited to droxidopa, levodopa, melevodopa, and etilevodopa.

In certain embodiments, the compounds provided herein can be combined with one or more L-dopa metabolism suppressors known in the art, including, but not limited to, carbidopa, benserazide, tolcapone, and entacapone.

In certain embodiments, the compounds provided herein can be combined with adamantine-based agents known in the art, including, but not limited to, amantadine, memantine, and rimantadine.

In certain embodiments, the compounds disclosed herein can be combined with one or more serotonin-norepinephrine reuptake inhibitors (SNRIs) known in the art, including, but not limited to bicifadine, desvenlafaxine, duloxetine, milnacipran, nefazodone, and venlafaxine.

In certain embodiments, the compounds disclosed herein can be combined with one or more selective serotonin reuptake inhibitors (SSRIs) known in the art, including, but not limited to alaproclate, citalopram, dapoxetine, escitalopram, etoperidone, fluoxetine, fluvoxamine, paroxetine, sertraline, and zimelidine.

In certain embodiments, the compounds disclosed herein can be combined with one or more acetylcholinesterase inhibitors known in the art, including, but not limited to metrifonate, physostigmine, neostigmine, pyridostigmine, ambenonium, demarcarium, rivastigmine, galantamine, donepezil, tacrine, and edrophonium.

In certain embodiments, the compounds disclosed herein can be combined with one or more tricyclic and tetracyclic antidepressants (TCAs) known in the art, including, but not limited to clomipramine, nefazodone, trazodone, amitriptyline, amoxapine, butriptyline, desipramine/lofepramine, dibenzepin, dothiepin, doxepin, imipramine, iprindole, melitracen, nortriptyline, opipramol, protriptyline, trimipramine, maprotiline and amineptine.

In certain embodiments, the compounds provided herein can be combined with one or more barbituates known in the art, including, but not limited to, allobarbital, alphenal, amobarbital, aprobarbital, barbexaclone, barbital, brallobarbital, brophebarbital, bucolome, butabarbital, butalbital, butobarbital, butallylonal, crotylbarbital, cyclobarbital, cyclopal, enallylpropymal, ethallobarbital, febarbamate, heptabarbital, hexethal, hexobarbital, mephobarbital, metharbital, methohexital, methylphenobarbital, narcobarbital, nealbarbital, pentobarbital, phenobarbital, phetharbital, prazitone, probarbital, propallylonal, proxibarbal, roxibarbital, reposal, secbutabarbital, secobarbital, sigmodal, spirobarbital, talbutal, thialbarbital, thiamylal, thiobarbital, thiobutabarbital, thiopental, valofane, vinbarbital, and vinylbital.

In certain embodiments, the compounds disclosed herein can be combined with one or more benzodiazepines (“minor tranquilizers”) known in the art, including, but not limited to alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

In certain embodiments, the compounds disclosed herein can be combined with one or more amphetamine-like stimulants known in the art, including, but not limited to the group including 4-bromomethcathinone, 4-fluoroamphetamine, 4-fluoromethamphetamine, 4-fluoromethcathinone, 4-methylmethcathinone, aletamine, amfepentorex, amphechloral, racemic amphetamine salts (dextroamphetamine, Adderall), amphetaminil, benzphetamine, bupropion, cathinone, chlorphentermine, clenbuterol, clobenzorex, clortermine, diethylpropion, dimethoxyamphetamine, dimethylamphetamine, dimethylcathinone, ephedrine, epinephrine, ethcathinone, ethylamphetamine, fenethylline, fenfluramine, fenproporex, fludorex, furfenorex, levomethamphetamine, misdexamfetamine, MDMA, mefenorex, methamphetamine, methcathinone, methoxyphedrine, methylone, octopamine, ortetamine, parahydroxyamphetamine, PCA, PIA, PMA, PMEA, PMMA, PPAP, phendimetrazine, phenmetrazine, phentermine, phenylephrine, phenylpropanolamine, propylamphetamine, pseudoephedrine, selegiline, synephrine, tiflorex, and xylopropamine.

The compounds disclosed herein can also be administered in combination with other classes of compounds, including, but not limited to, sepsis treatments, such as drotrecogin-α; steroidals, such as hydrocortisone; local or general anesthetics, such as ketamine; platelet aggregation inhibitors, such as clopidogrel; HMG-CoA reductase inhibitors (statins), such as atorvastatin; anticoagulants, such as heparin; thrombolytics, such as streptokinase; fibrates, such as clofibrate; bile acid sequestrants, such as colestipol; non-steroidal anti-inflammatory agents (NSAIDs), such as naproxen; cholesteryl ester transfer protein (CETP) inhibitors, such as anacetrapib; anti-bacterial agents, such as ampicillin; anti-fungal agents, such as amorolfine; norepinephrine reuptake inhibitors (NRIs), such as atomoxetine; dopamine reuptake inhibitors (DARIs), such as methylphenidate; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; squalene synthetase inhibitors; fibrates; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, tricrynafen, chlorthalidone, furosenilde, musolimine, bumetanide, triamterene, amiloride, and spironolactone; recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunomide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating a hormone-mediated disorder and/or a pigment-mediated disorder in a human or animal subject in need of such treatment comprising administering to said subject an amount of a compound disclosed herein effective to reduce or prevent said disorder in the subject, in combination with at least one additional agent for the treatment of said disorder. In a related aspect, certain embodiments provide therapeutic compositions comprising at least one compound disclosed herein in combination with one or more additional agents for the treatment of a hormone-mediated disorder and/or a pigment-mediated disorder.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in Renault et al., Organic Letters 2004, 6(3), 397-400; Davis B, J of Labelled Compounds and Radiopharmaceuticals 1987, 24(2), 199-204; Hopfgartner et al., J. Mass. Spectrom. 1996, 31, 69-76; Kendall J, J. Labelled Cpd. Radiopharm. 2000, 43, 917-924; Humphrey et al., Organic Process Research & Development 2007, 11, 1069-1075, and references cited therein and routine modifications thereof. Compounds as disclosed herein can also be prepared as shown in any of the following schemes and routine modifications thereof.

The following schemes can be used to practice the present invention. Any position shown as hydrogen can be optionally substituted with deuterium.

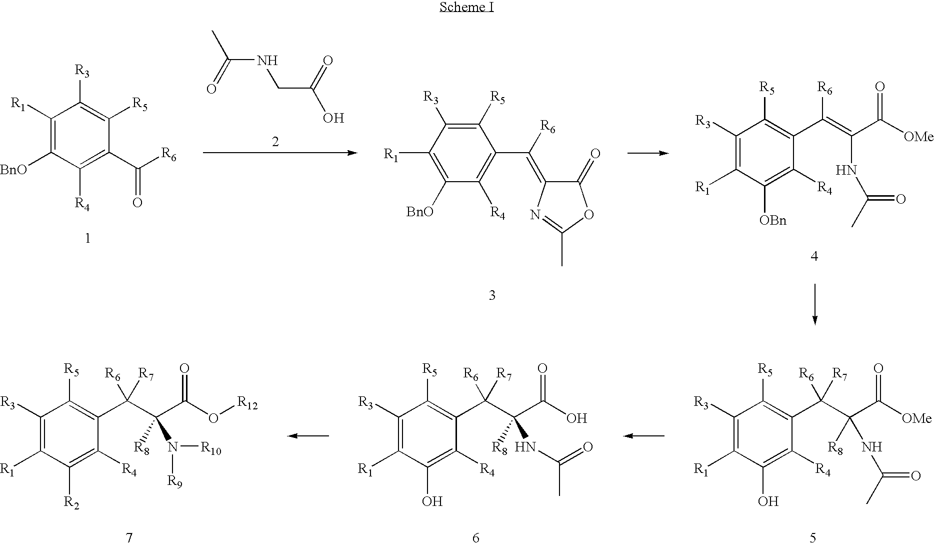

Compound 1 is reacted with compound 2 in an appropriate solvent, such as acetic anhydride, in the presence of an appropriate base, such as sodium acetate, at an elevated temperature to give compound 3. Compound 3 is treated with an appropriate base, such as sodium acetate, in an appropriate solvent, such as methanol, to afford compound 4. Compound 4 is reacted with an appropriate reducing agent, such as hydrogen gas and palladium on carbon, in an appropriate solvent, such as methanol, at an elevated temperature and pressure to give compound 5. Compound 5 is treated with an appropriate enzyme, such as Alcalase®, in an appropriate buffer, such as a phosphate buffer, to give compound 6. Compound 6 is treated with an appropriate acid, such as hydrochloric acid, in an appropriate solvent, such as methanol, at an elevated temperature to afford compound 7 (wherein R₂ is a hydroxyl group) of Formula I.

Deuterium can be incorporated to different positions synthetically, according to the synthetic procedures as shown in Scheme I, by using appropriate deuterated intermediates. For example, to introduce deuterium at one or more positions of R₁ and R₃-R₆, compound 1 with the corresponding deuterium substitutions can be used. To introduce deuterium at one or more positions of R₇ and R₈, deuterium gas can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as N—H and O—H groups, via proton-deuterium equilibrium exchange. To introduce deuterium at R₂, R₉, R₁₀, or R₁₂, these protons may be replaced with deuterium selectively or non-selectively through a proton-deuterium exchange method known in the art.

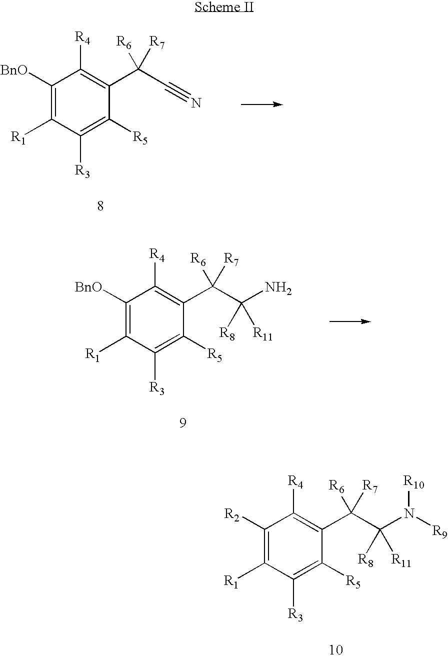

Compound 8 is treated with an appropriate reducing agent, such as lithium aluminum hydride, in an appropriate solvent, such as tetrahydrofuran, to give compound 9. Compound 9 is then treated with an appropriate reducing agent, such as hydrogen gas and palladium on carbon, in the presence of an appropriate acid, such as hydrochloric acid, in an appropriate solvent, such as ethanol, and at an elevated temperature to give compound 10 (wherein R₂ is a hydroxyl group) of Formula I.

Deuterium can be incorporated to different positions synthetically, according to the synthetic procedures as shown in Scheme II, by using appropriate deuterated intermediates. For example, to introduce deuterium at one or more positions of R₁, R₃-R₇, compound 8 with the corresponding deuterium substitutions can be used. To introduce deuterium at one or more positions of R₈ and R₁₁, lithium aluminum deuteride can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as N—H and O—H groups, via proton-deuterium equilibrium exchange. To introduce deuterium at R₂, R₉, or R₁₀, these protons may be replaced with deuterium selectively or non-selectively through a proton-deuterium exchange method known in the art.

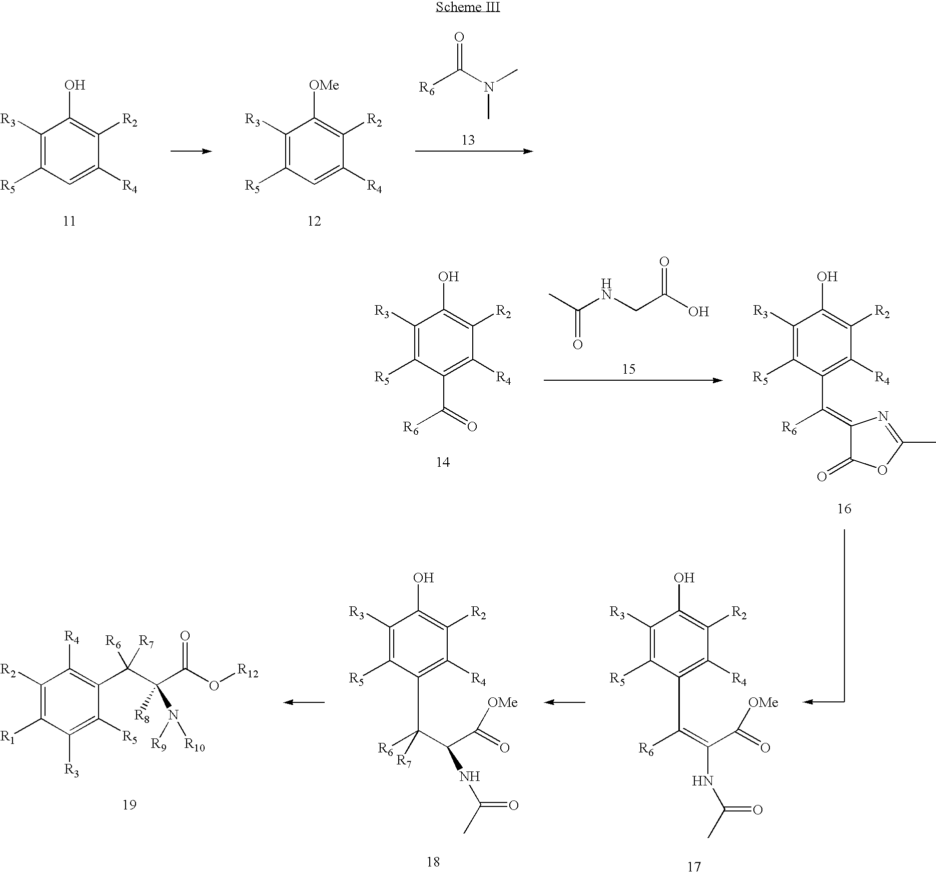

Compound 11 is reacted with an appropriate alkylating agent, such as iodomethane, in the presence of an appropriate base, such as sodium hydride, in an appropriate solvent, such as tetrahydrofuran, at an elevated temperature to give compound 12. Compound 12 is reacted with compound 13 and an appropriate chlorinating agent, such as diphosphoryl chloride, at an elevated temperature to give compound 14. Compound 14 is reacted with compound 15 in the presence of an appropriate base, such as sodium acetate, in an appropriate solvent, such as acetic anhydride, at an elevated temperature to give compound 16. Compound 16 is treated with an appropriate base, such as sodium acetate, in an appropriate solvent, such as methanol, at an elevated temperature to give compound 17. Compound 17 is treated with an appropriate reducing agent, such as a combination of hydrogen gas and Knowles/Monsanto rhodium catalyst, in an appropriate solvent, such as a mixture of isopropyl alcohol and water, to afford compound 18. Compound 18 is treated with an appropriate acid, such as hydrochloric acid, to give compound 19 (wherein R₁ is a hydroxyl group) of Formula I.

Deuterium can be incorporated to different positions synthetically, according to the synthetic procedures as shown in Scheme III, by using appropriate deuterated intermediates. For example, to introduce deuterium at one or more positions of R₂-R₅, compound 11 with corresponding deuterium substitutions can be used. To introduce deuterium at R₆, compound 13 with corresponding deuterium substitutions can be used. To introduce deuterium at one or more positions of R₇ and R₈, deuterium gas can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as N—H and O—H groups, via proton-deuterium equilibrium exchange. To introduce deuterium at R₁, R₉, R₁₀, or R₁₂, these protons may be replaced with deuterium selectively or non-selectively through a proton-deuterium exchange method known in the art.

The invention is further illustrated by the following examples. All IUPAC names were generated using CambridgeSoft's ChemDraw 10.0.

The following compounds can generally be made using the methods described above. It is expected that these compounds when made will have activity similar to those described in the examples above.

### EXAMPLE 1

### 2-Amino-3-(3-hydroxy-phenyl)-propionic acid (L-m-tyrosine)

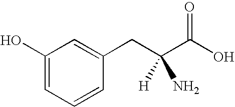

### Step 1

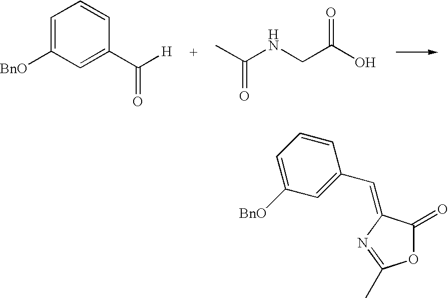

4-(3-Benzyloxy-benzylidene)-2-methyl-4H-oxazol-5-one: A mixture of 3-benzyloxy-benzaldehyde (20 g; 94.3 mmol), sodium acetate (14.2 g; 104.5 mmol), N-acetyl glycine (10.92 g; 93.3 mmol), and acetic anhydride (47 mL) was heated at about 115° C. in an oil bath for about 18 hours. The mixture was cooled to ambient temperature and used in the next step without further purification.

### Step 2

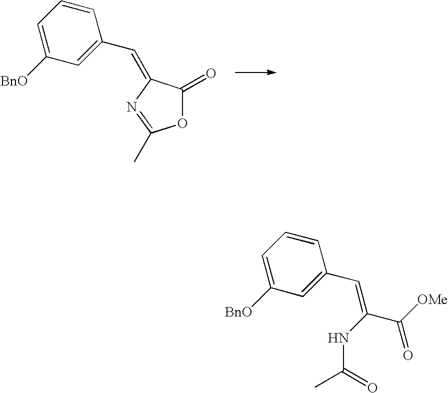

2-Acetylamino-3-(3-benzyloxy-phenyl)-acrylic acid methyl ester: The mixture from Example 1, Step 1 was poured into a solution of sodium acetate (15 g) and methanol (500 mL). The resulting mixture was stirred at ambient temperature for about 48 hours. Following standard extractive workup, the crude product was purified by silica gel column chromatography to give the title product as a white solid (20.3 g; 67% yield).

### Step 3

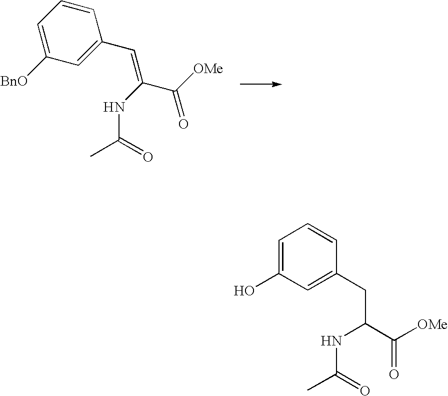

2-Acetylamino-3-(3-hydroxy-phenyl)-propionic acid methyl ester: A solution of 2-acetylamino-3-(3-benzyloxy-phenyl)-acrylic acid methyl ester (1.0 g; 3.08 mmol) dissolved in methanol-ethyl acetate (1:1, 60 mL) was hydrogenated in an H-Cube™ continuous-flow hydrogenation reactor (Thales Nanotechnology, Budapest, Hungary) equipped with a water reservoir for the generation of hydrogen gas, and a 10% palladium on carbon catalyst cartridge. The reactor was pressurized to 40 bar and heated to about 50° C., with a flow rate of 2 mL/min. The solvent was removed in vacuo to obtain the title product (0.72 g; 99% yield).

### Step 4

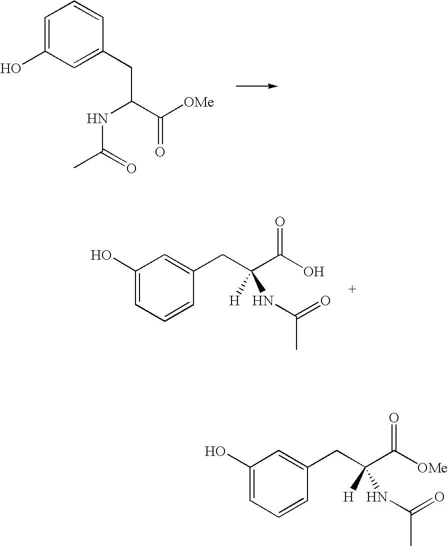

(S)-2-Acetylamino-3-(3-hydroxy-phenyl)-propionic acid, and (R)-2-Acetylamino-3-(3-hydroxy-phenyl)-propionic acid methyl ester: Alcalase® (1 mg) was added to 2-acetylamino-3-(3-hydroxyphenyl)-propionic acid methyl ester (1.25, 5.27 mmol) suspended in pH 7.5 phosphate buffer (20 mL). The mixture was stirred at ambient temperature for about 7 hours, while the pH was maintained at about 7.5, by adding 1N sodium hydroxide. Standard extractive workup with dichloromethane, gave (S)-2-acetylamino-3-(3-hydroxy-phenyl)-propionic acid methyl ester in the aqueous phase, and (R)-2-acetylamino-3-(3-hydroxyphenyl)-propionic acid methyl ester in the organic phase. (S)-2-Acetylamino-3-(3-hydroxyphenyl)-propionic acid was used in the next step without any further purification.

### Step 5

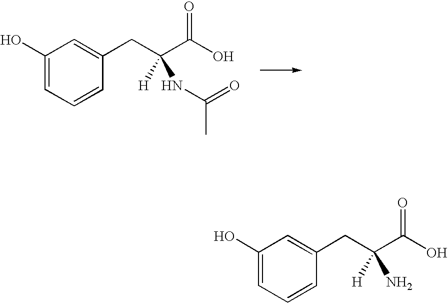

2-Amino-3-(3-hydroxy-phenyl)-propionic acid (L-m-Tyrosine): A solution of (S)-2-acetylamino-3-(3-hydroxy-phenyl)-propionic acid in methanol was treated with concentrated hydrochloric acid (4 mL) at about 100° C. for about 2 hours. The resulting solution was cooled to ambient temperature, and the pH was adjusted to about 6 by adding 4N sodium hydroxide. The resulting precipitate was filtered, washed with water, and dried to provide the title compound as a solid (0.33 g; 69% yield). ¹H-NMR (D₂O+trace DCl): 7.05 (t, 1H, J=7.8 Hz), 6.60 (m, 3H), 4.12 (m, 1H), 1.88-3.08 (m, 2H). MS: 182.2 (M+1).

### EXAMPLE 2

### d₃-2-Amino-3-(3-hydroxy-phenyl)-propionic acid (L-m-d₂-tyrosine)

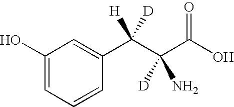

### Step 1

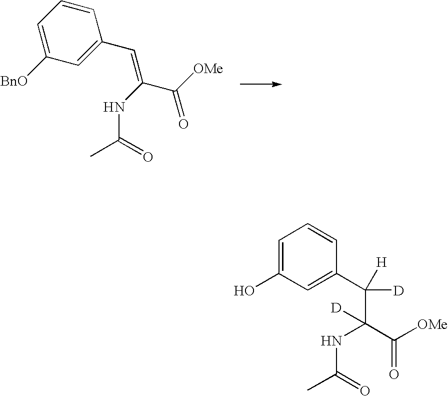

d₂-2-Acetylamino-3-(3-hydroxy-phenyl)-propionic acid methyl ester: The procedure of Example 1, Step 3 was followed, but substituting deuterium oxide for water, and dₗ-methanol for methanol. The title product was isolated as a solid (98% yield). ¹H-NMR (DMSO-d₆) δ: 9.28 (s, 1H), 8.30 (s, 1H), 7.06 (m, 1H), 6.62 (m, 3H), 3.59 (s, 3H), 1.80 (s, 3H).

### Step 2

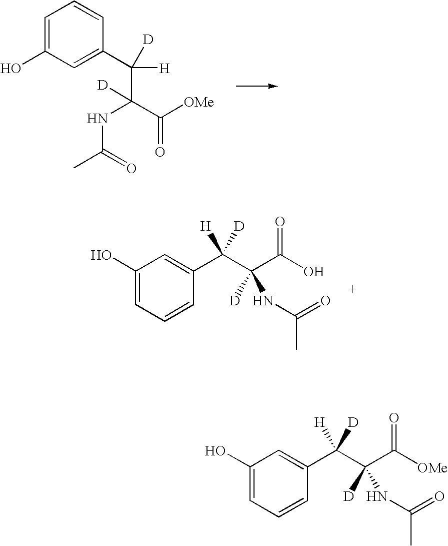

(S,S)-d₂-2-Acetylamino-3-(3-hydroxy-phenyl)-propionic acid, and (R,R)-d₂-2-acetylamino-3-(3-hydroxy-phenyl)-propionic acid methyl ester: The procedure of Example 1, Step 4 was followed, but substituting d₂-2-acetylamino-3-(3-hydroxy-phenyl)-propionic acid methyl ester for 2-acetylamino-3-(3-hydroxy-phenyl)-propionic acid methyl ester. (S)-d₂-2-Acetylamino-3-(3-hydroxy-phenyl)-propionic acid was used in the next step without any further purification.

### Step 3

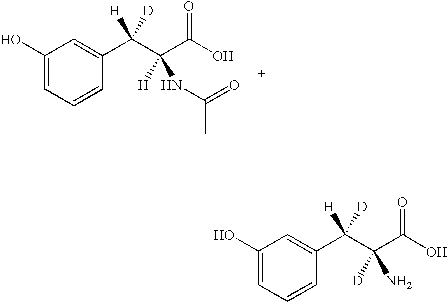

d₂-2-Amino-3-(3-hydroxy-phenyl)-propionic acid (L-m-d₂-Tyrosine): The procedure of Example 1, Step 5 was followed, but substituting (S,S)-d₂-2-acetylamino-3-(3-hydroxy-phenyl)-propionic acid for (S)-2-acetylamino-3-(3-hydroxy-phenyl)-propionic acid. The title product was isolated as a solid (330 mg; yield 69%). ¹H-NMR (D₂O+trace DCl) δ: 7.10 (m, 1H), 6.63 (m, 3H), 2.97 (s, 1H). MS: 184.2 (M+H).

### EXAMPLE 3

d₃-2-Amino-3-(3-hydroxy-phenyl)-propionic acid (L-m-d₃-tyrosine)

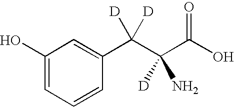

### Step 1

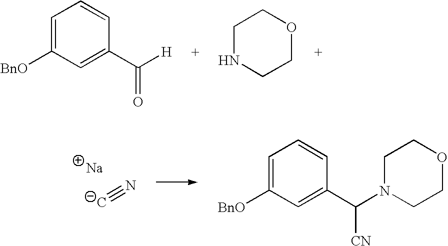

3-Benzyloxyphenyl)-morpholin-4-yl-acetonitrile: At about 0° C., perchloric acid (70%, 4.75 mL) was added dropwise to a stirred solution of morpholine (10 mL). 3-Benzyloxybenzaldehyde (11.66 g, 55 mmol) was then added, and the resulting mixture was heated at about 70° C. for about 4 hours. A solution of sodium cyanide (3.9 g, 79.6 mmol) was dissolved in water (2.5 mL) and then added to the mixture. After heating the mixture to about 70° C. for about 1 hour, the mixture was poured into ice-water. Following standard extractive workup with ethyl acetate, the crude product was purified by recrystallization from isopropanol to afford the title product (14.7 g; 87% yield). ¹H-NMR (CDCl₃) δ: 7.45-6.85 (m, 9H), 5.01 (s, 2H), 4.78 (s, 1H), 3.71 (m, 4H), 2.57 (m, 4H).

### Step 2

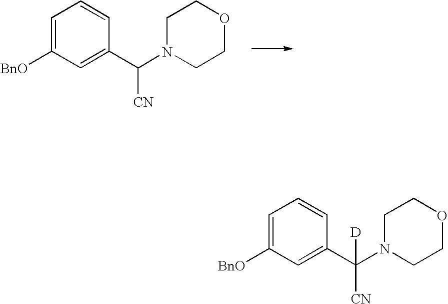

d₁-(3-Benzyloxyphenyl)-morpholin-4-yl-acetonitrile: 95% Sodium hydride (1.33 g, 50 mmol) was added to 3-(benzyloxyphenyl)-morpholin-4-yl-acetonitrile (7.7 g, 25 mmol) dissolved in tetrahydrofuran (40 mL). The resulting mixture was heated at about 40° C. for about 16 hours and then cooled to ambient temperature. The pH of the mixture was adjusted to 1-2 by adding dᵢ-hydrochloric acid in deuterium oxide (10 mL). After stirring for 10 minutes, standard extractive workup with ethyl acetate yielded the title product (7.7 g). ¹H-NMR (CDCl₃) δ: 7.45-6.85 (m, 9H), 5.01 (s, 2H), 3.71 (m, 4H), 2.57 (m, 4H).

### Step 3

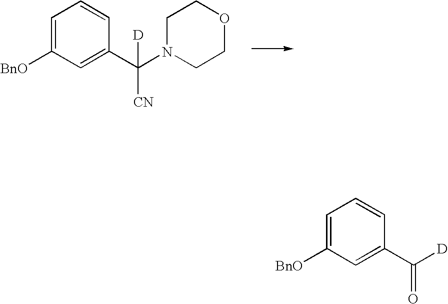

d₁-3-Benzyloxybenzaldehyde: At about 100° C., (3-benzyloxyphenyl)-morpholin-4-yl-acetonitrile (7.7 g, 25 mmol) was treated with 2N hydrochloric acid for about 16 hours. Standard extractive workup with ethyl acetate afforded the title product (73%; 3.9 g). ¹H-NMR (CDCl₃) δ: 7.45-6.85 (m, 9H), 5.01 (s, 2H).

### Step 4

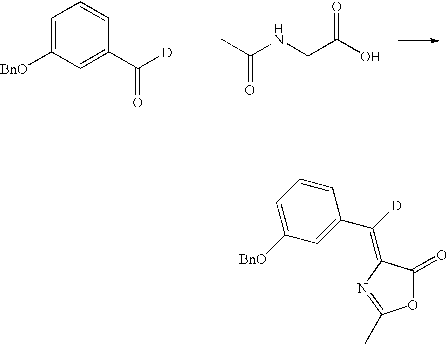

d₁-4-(3-Benzyloxybenzylidene)-2-methyl-4H-oxazol-5-one: The title product was made by following the procedure set forth in Example 1, Step 1, but substituting dᵢ-3-benzyloxy-benzaldehyde for 3-benzyloxybenzaldehyde. The title product was used in the next step without further purification.

### Step 5

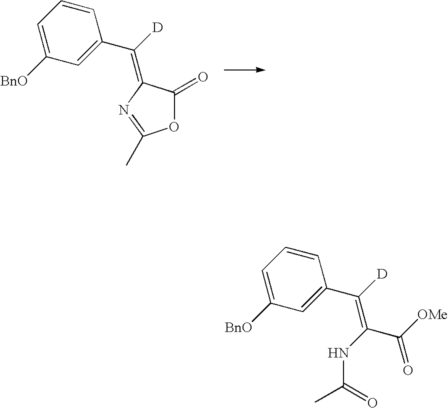

d₁-2-Acetylamino-3-(3-benzyloxyphenyl)-acrylic acid methyl ester: The title product was made by following the procedure set forth in Example 1, Step 2, but substituting d₁-4-(3-benzyloxybenzylidene)-2-methyl-4H-oxazol-5-one for 4-(3-benzyloxybenzylidene)-2-methyl-4H-oxazol-5-one. The title product was isolated as a solid (65% yield). ¹H-NMR (DMSO-d₆) δ: 9.86 (s, 1H), 7.01-7.50 (m, 9H), 5.13 (s, 2H), 3.70 (s, 3H), 1.98 (s, 3H).

### Step 6

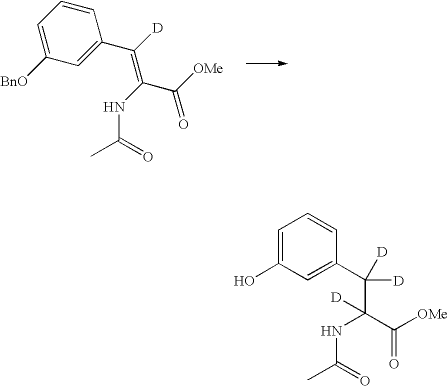

d₃-2-Acetylamino-3-(3-hydroxyphenyl)-propionic acid methyl ester: The title product was made by following the procedure set forth in Example 2, Step 1, but substituting d₁-2-acetylamino-3-(3-benzyloxyphenyl)-acrylic acid methyl ester for 2-acetylamino-3-(3-benzyloxyphenyl)-acrylic acid methyl ester. The title product was isolated as a solid (82% yield). ¹H-NMR (DMSO-d₆) δ: 9.29 (s, 1H), 8.25 (s, 1H), 7.06 (m, 1H), 6.60 (m, 3H), 3.59 (s, 3H), 1.80 (s, 3H).

### Step 7

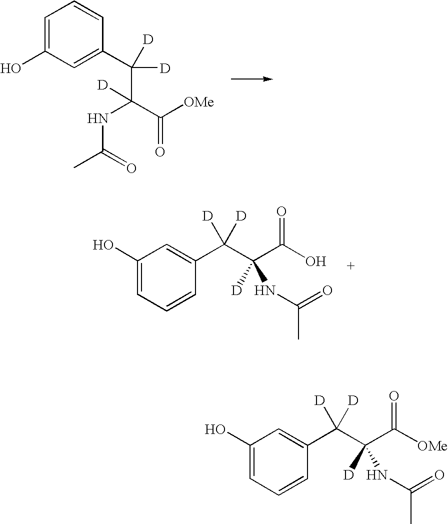

(S)-d₃-2-Acetylamino-3-(3-hydroxyphenyl)-propionic acid, and (R)-d₃-2-Acetylamino-3-(3-hydroxyphenyl)-propionic acid methyl ester: The title product was made by following the procedure set forth in Example 1, Step 4, but substituting d₃-2-acetylamino-3-(3-hydroxyphenyl)-propionic acid methyl ester for 2-acetylamino-3-(3-hydroxyphenyl)-propionic acid methyl ester.

### Step 8

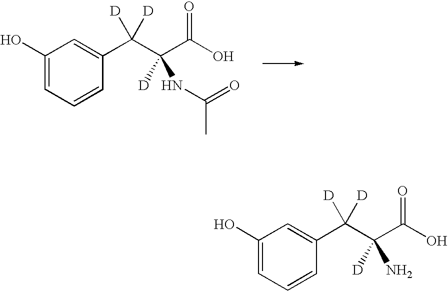

d₃-2-Amino-3-(3-hydroxy-phenyl)-propionic acid (L-m-d₃-Tyrosine): The title product was made by following the procedure set forth in Example 1, step 5, but substituting d₃-(S)-2-acetylamino-3-(3-hydroxyphenyl)-propionic acid for (S)-2-acetylamino-3-(3-hydroxyphenyl)-propionic acid. The title product was isolated as a solid (39% yield). ¹H-NMR (D₂O+trace DCl) δ: 7.10 (m, 1H), 6.63 (m, 3H) MS: 185.2 (M+H).

### EXAMPLE 4

### 3-(2-Amino-ethyl)-phenol hydrochloride salt (m-tyramine)

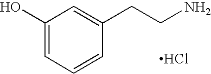

### Step 1

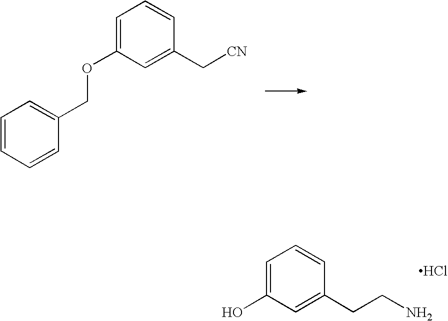

3-(2-Amino-ethyl)-phenol (m-tyramine): A solution of (3-benzyloxy-phenyl)-acetonitrile (112 mg, 0.5 mmol) dissolved in methanol (40 mL) was hydrogenated in an H-Cube™ continuous-flow hydrogenation reactor (Thales Nanotechnology, Budapest, Hungary) equipped with a water reservoir for the generation of hydrogen gas, and a Raney Ni catalyst cartridge. The reactor was pressurized to 60 bar and heated to about 70° C., with a flow rate of 2 mL/min. The solvent was removed, and the resulting residue was treated with ethyl acetate (5 mL) containing 2N hydrochloric acid in ether (0.5 mL) at ambient temperature for about 10 minutes. The resulting precipitate was collected by filtration and washed with ether to afford the title compound as a hydrochloride salt (40 mg; 49%). ¹H-NMR (MeOD) δ: 7.17 (m, 1H), 6.69 (m, 3H), 3.15 (t, 2H, J=8.1 Hz), 2.88 (t, 2H, J=8.1 Hz) MS: 138.3 (M+H).

### EXAMPLE 5

### d₄-3-(2-Amino-ethyl)-phenol hydrochloride salt (m-d₄-tyramine)

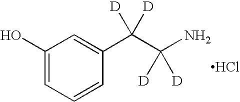

### Step 1

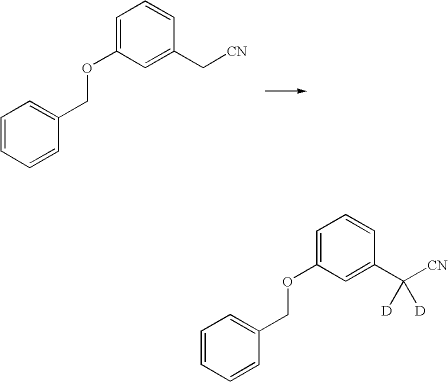

d₂-(3-Benzyloxy-phenyl)-acetonitrile: A solution of (3-benzyloxyphenyl)-acetonitrile (0.45 g, 2.0 mmol), potassium carbonate (0.83 g, 6.0 mmol), deuterium oxide (10 mL), and dioxane (0.5 mL) was heated at about 100° C. for about 24 hours. After cooling to ambient temperature, standard extractive workup with ethyl acetate afforded the title product as a solid (405 mg; 90% yield). ¹H-NMR (CDCl₃) δ: 7.70-6.90 (m, 9H), 5.08 (s, 3H).

### Step 2

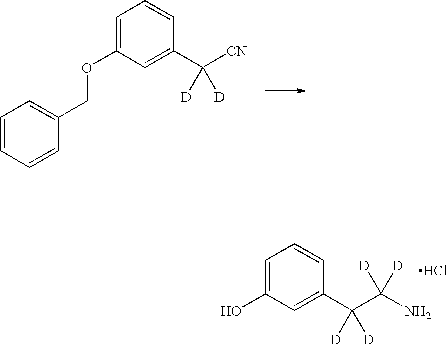

d₄-3-(2-Amino-ethyl)-phenol (m-d₄-tyramine): The title compound was made by following the procedure set forth in Example 4, Step 1, but substituting dᵢ-methanol for methanol, and deuterium oxide for water. The title compound is isolated as a solid (56% yield). ¹H-NMR (CD₃OD) δ: 7.17 (m, 1H), 6.69 (m, 3H), MS: 142.3 (M+H).

The following compounds can generally be made using the methods described above. It is expected that these compounds when made will have activity similar to those described in the examples above:

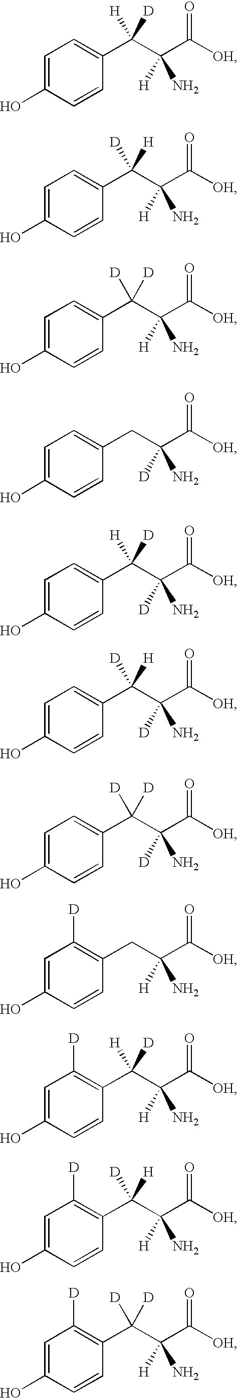

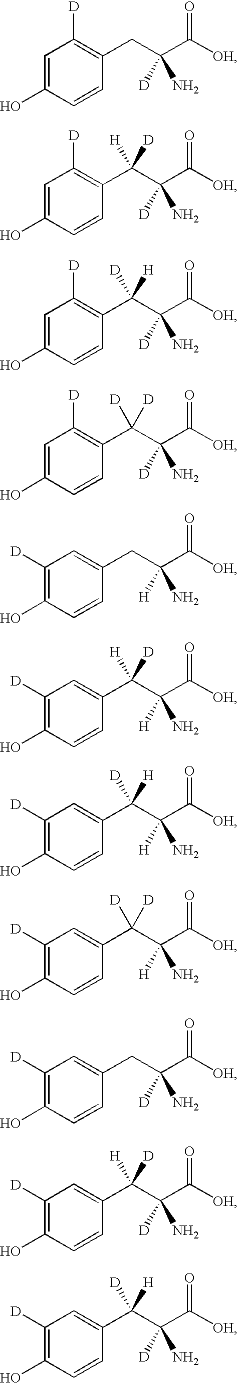

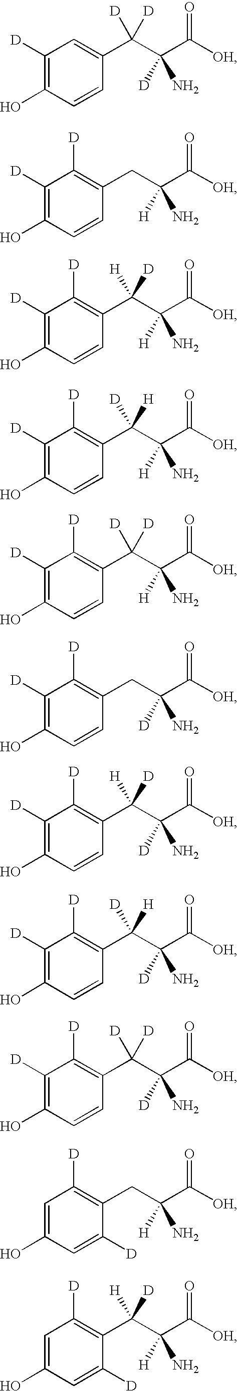

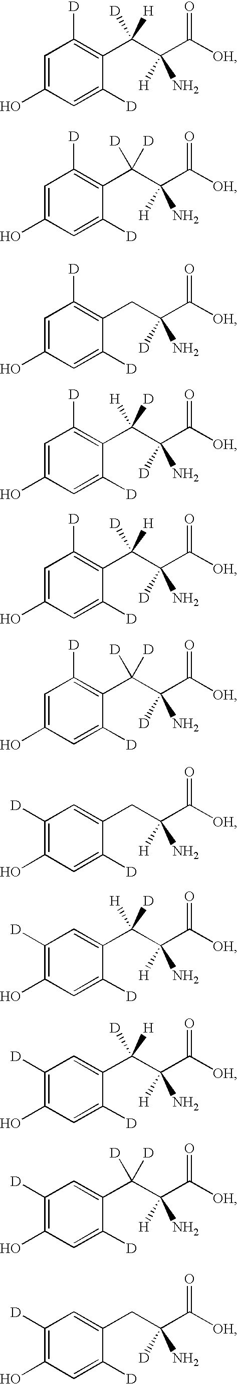

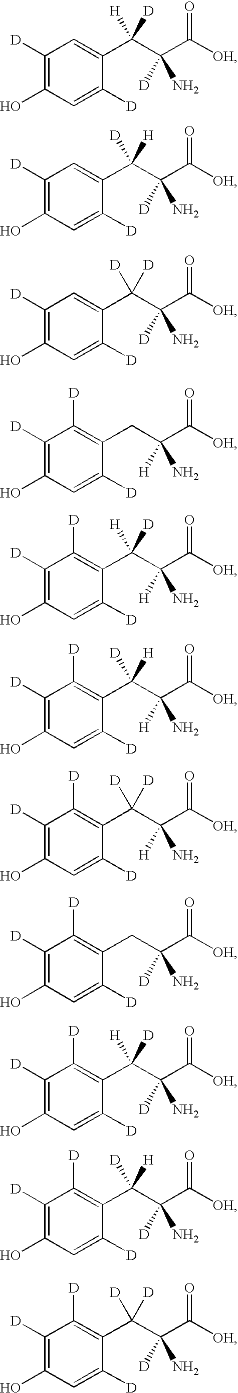

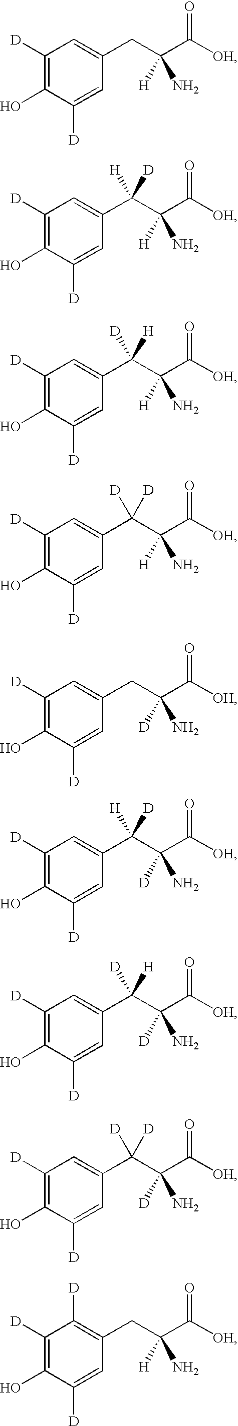

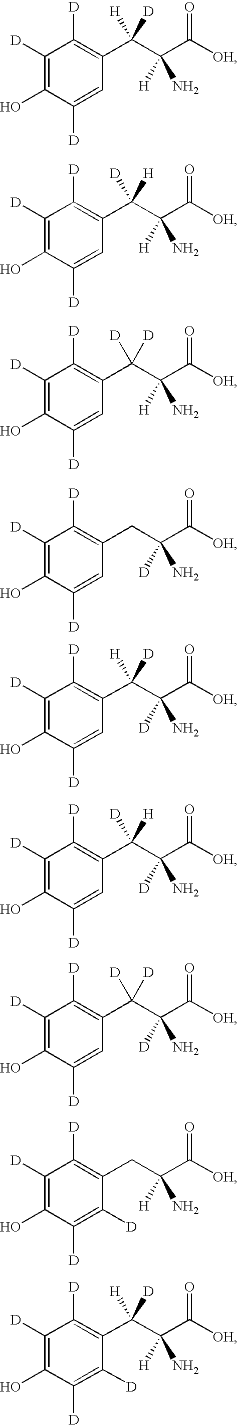

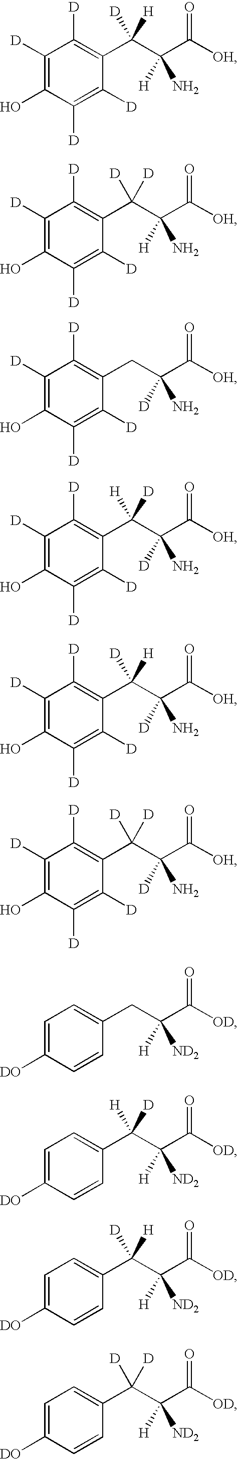

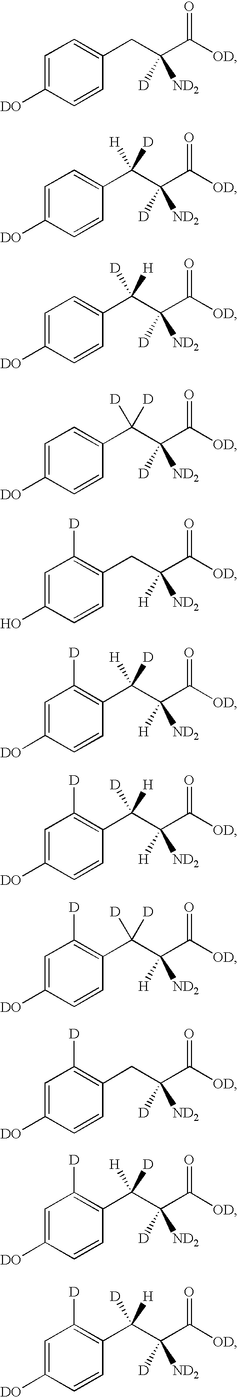

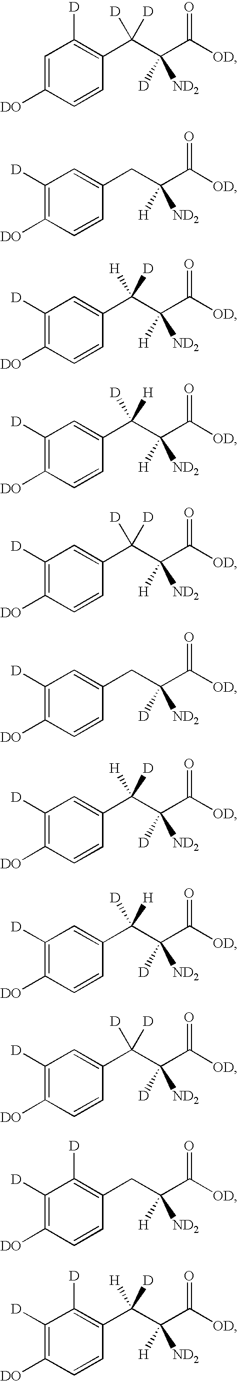

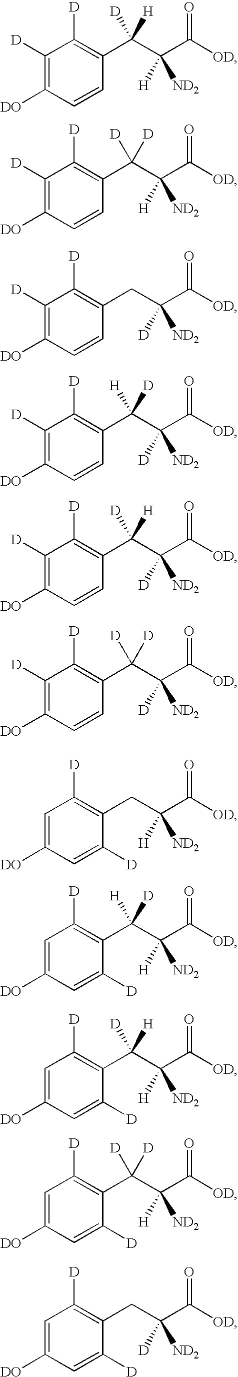

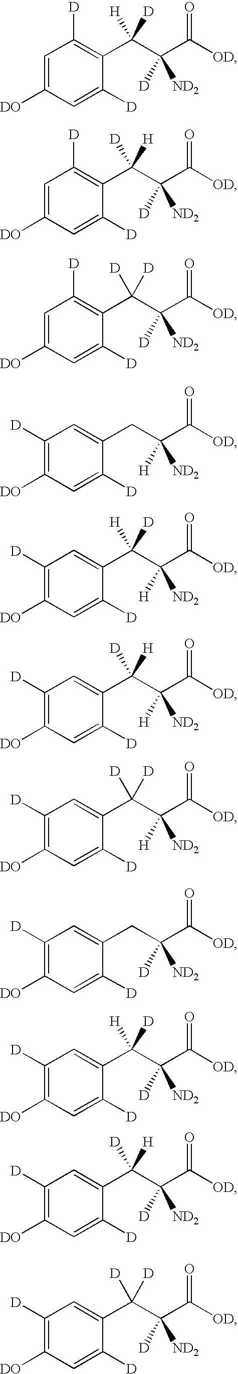

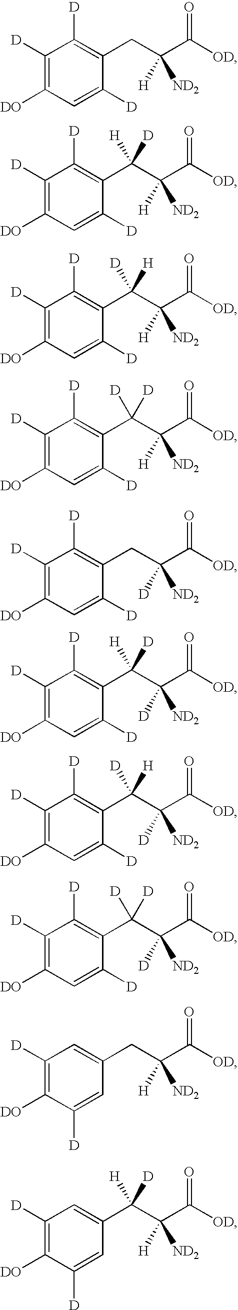

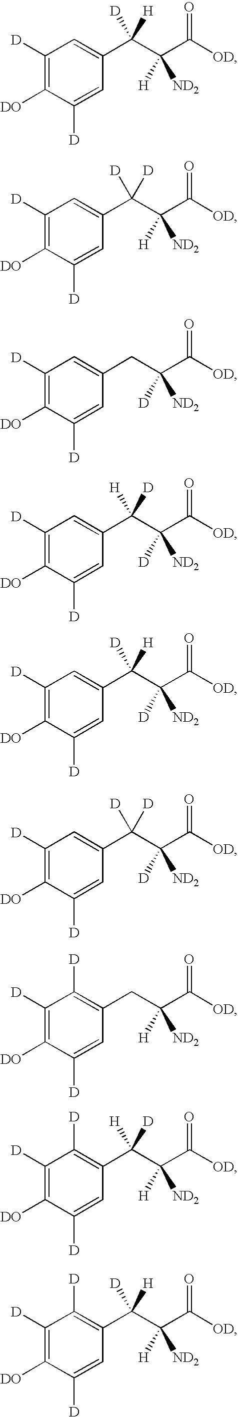

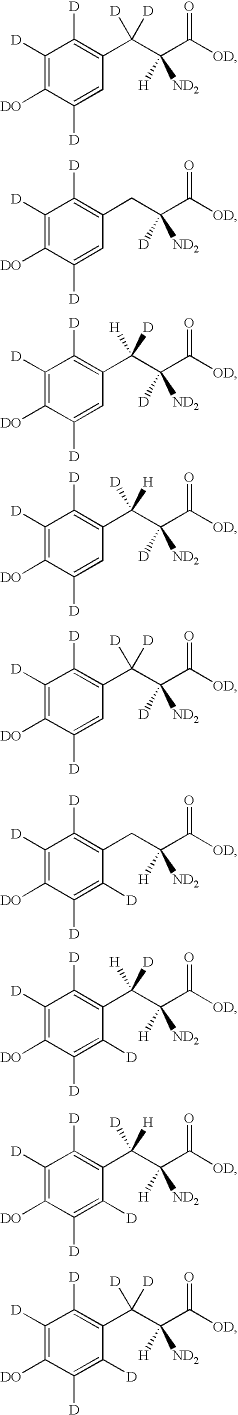

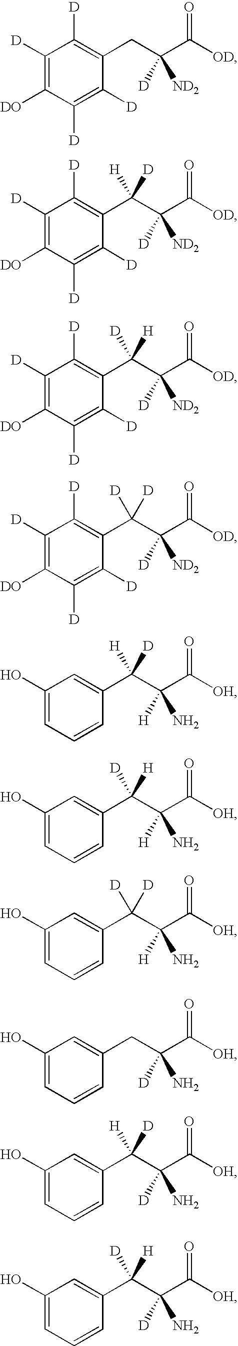

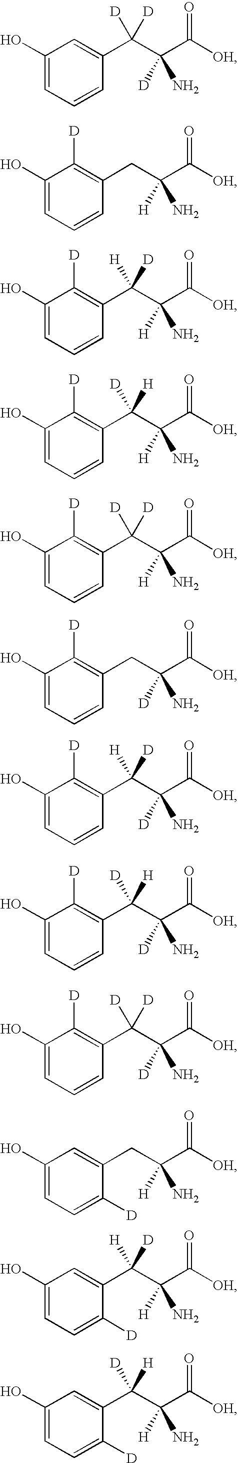

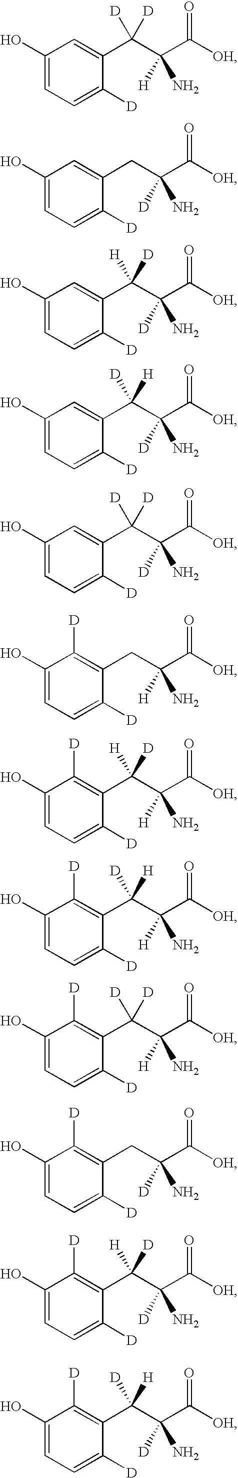

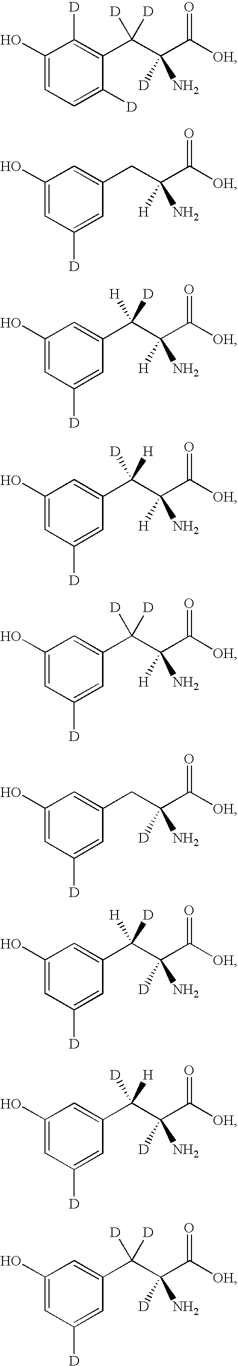

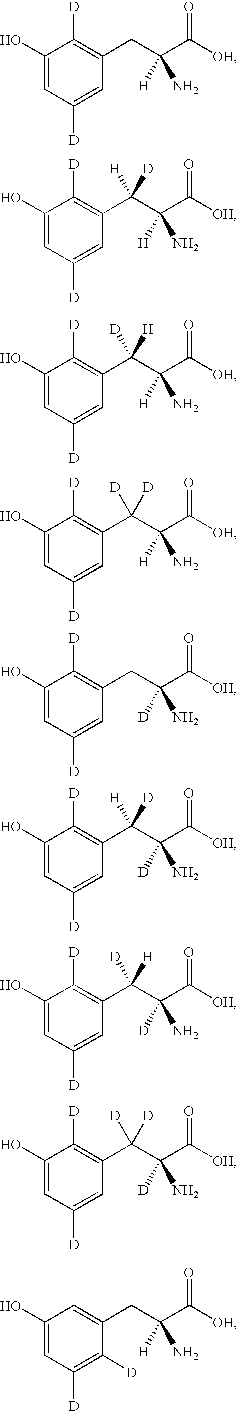

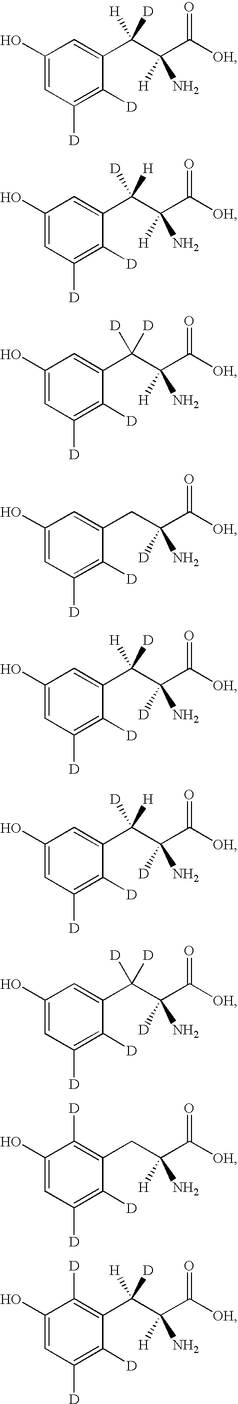

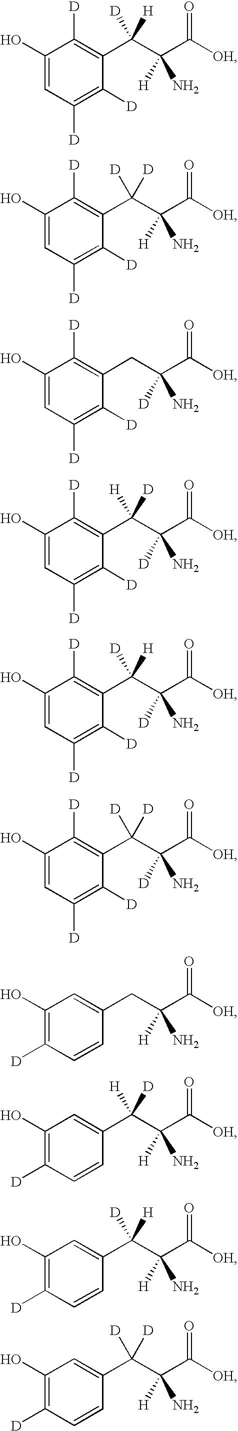

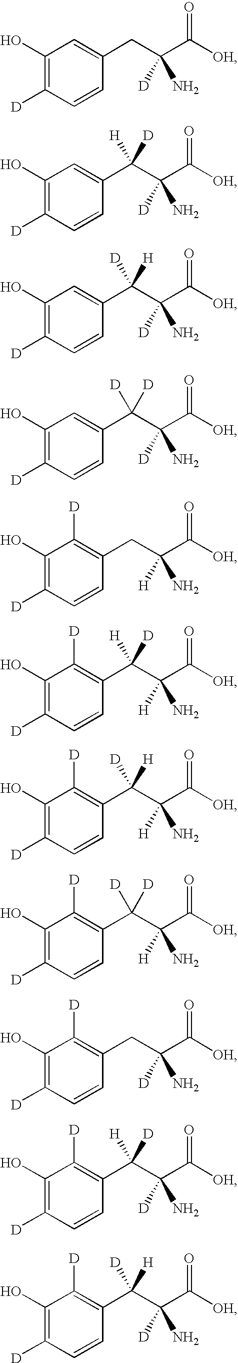

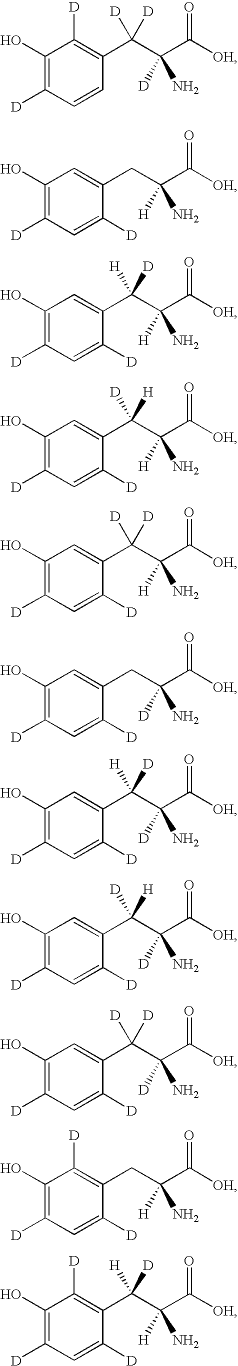

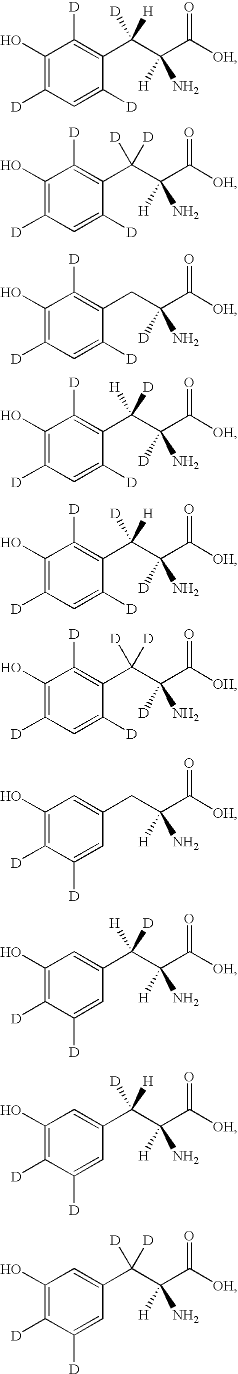

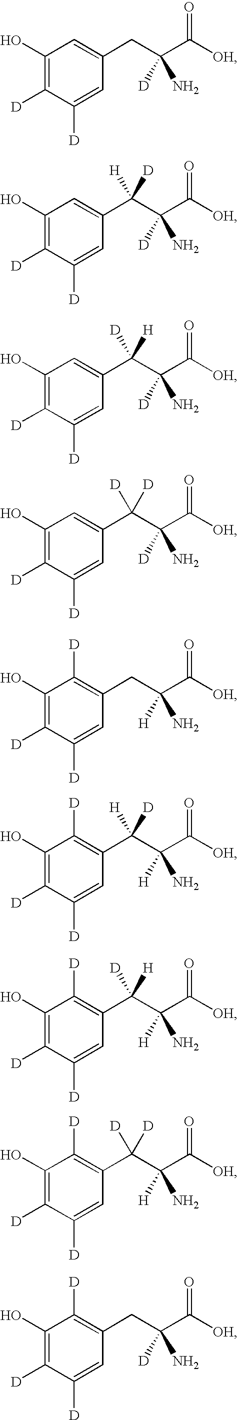

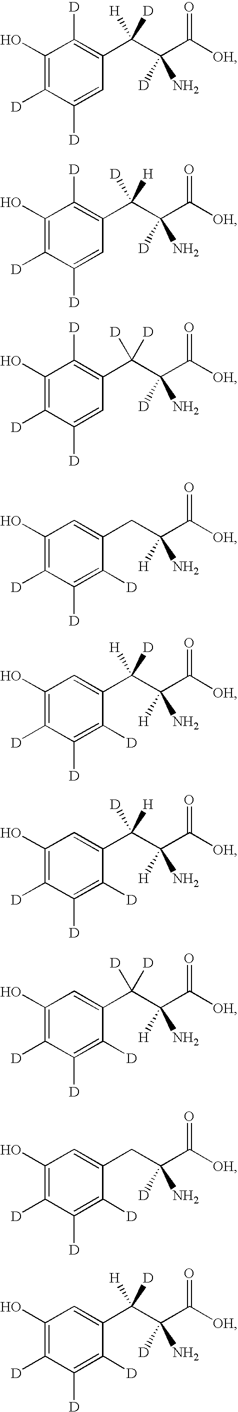

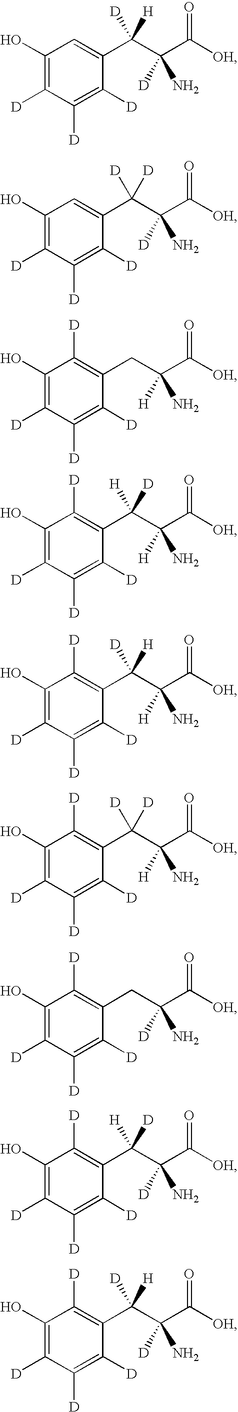

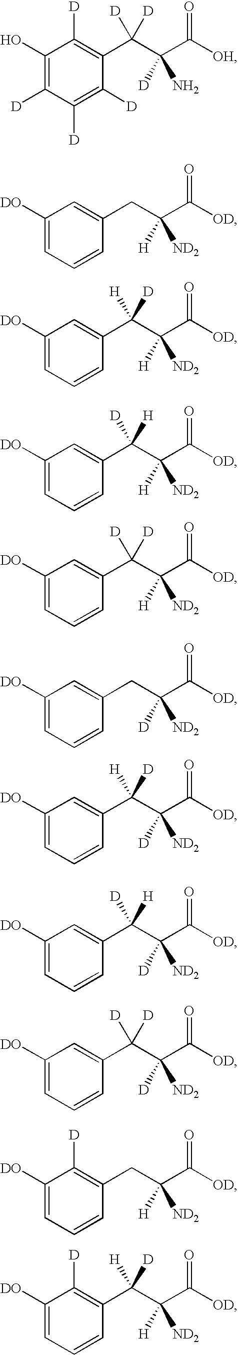

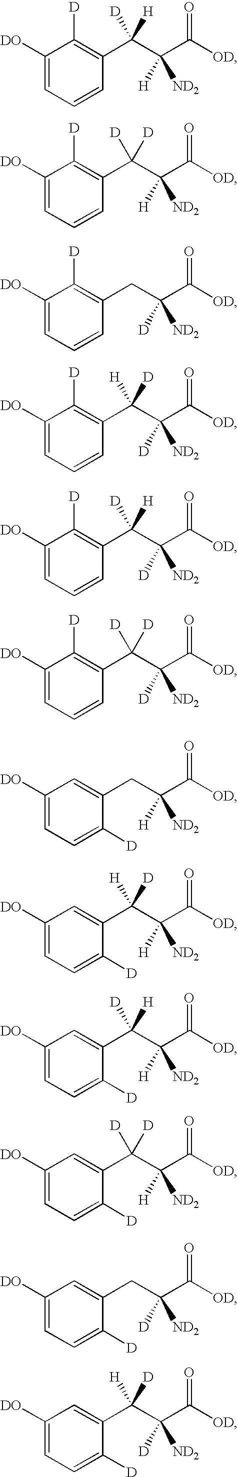

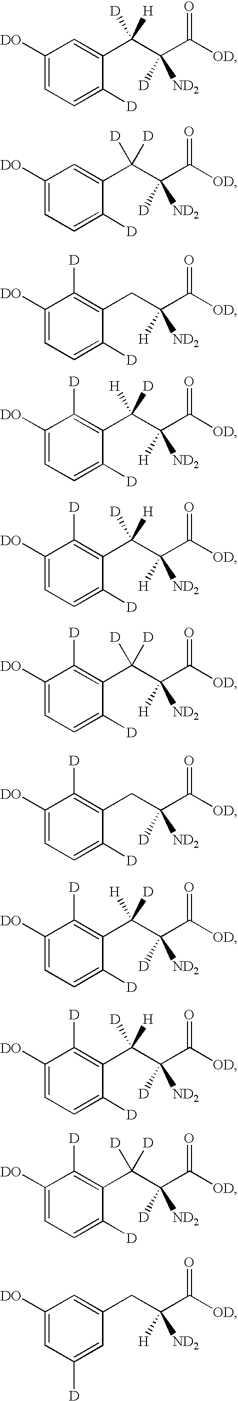

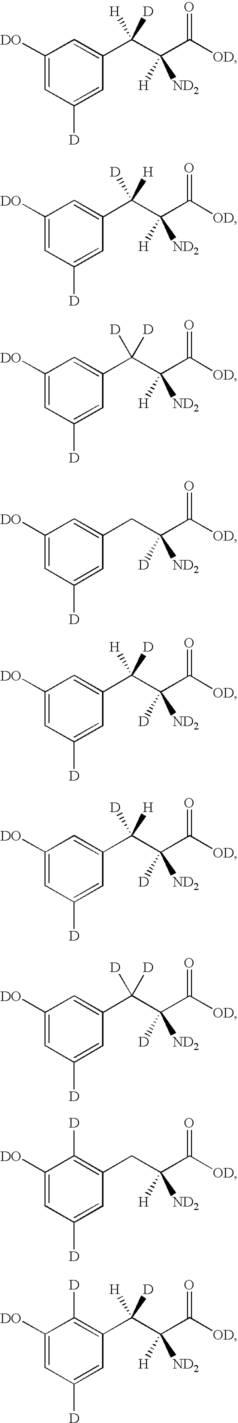

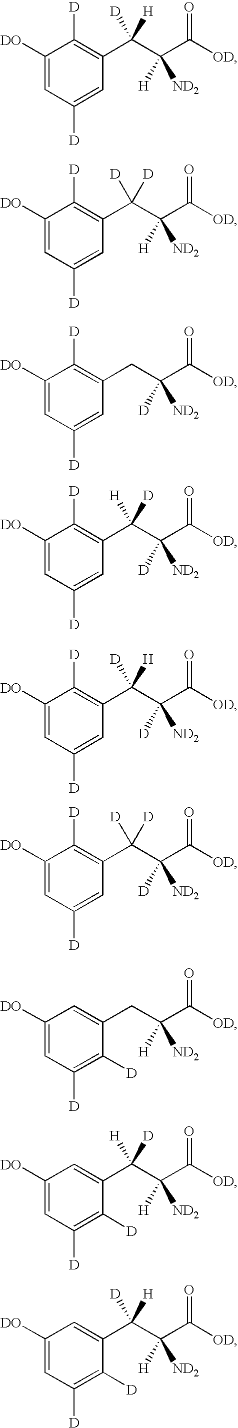

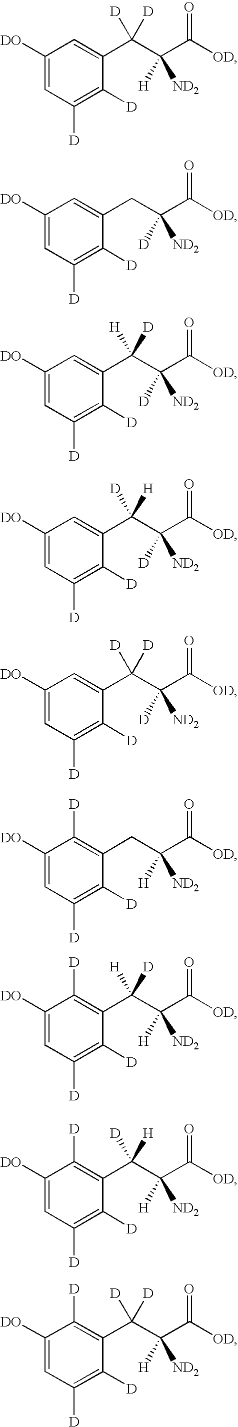

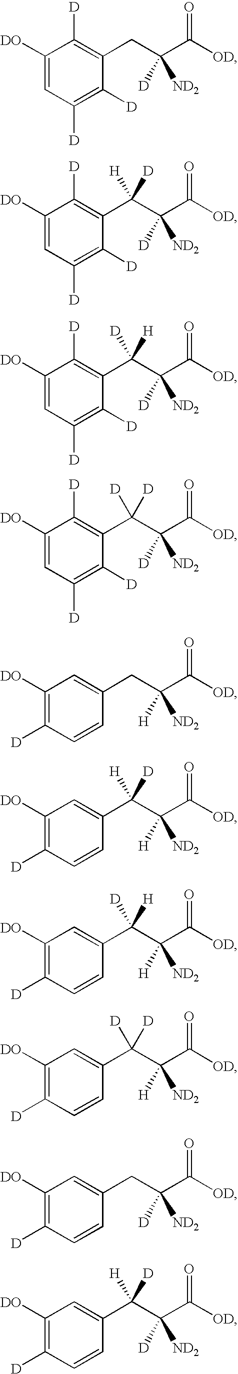

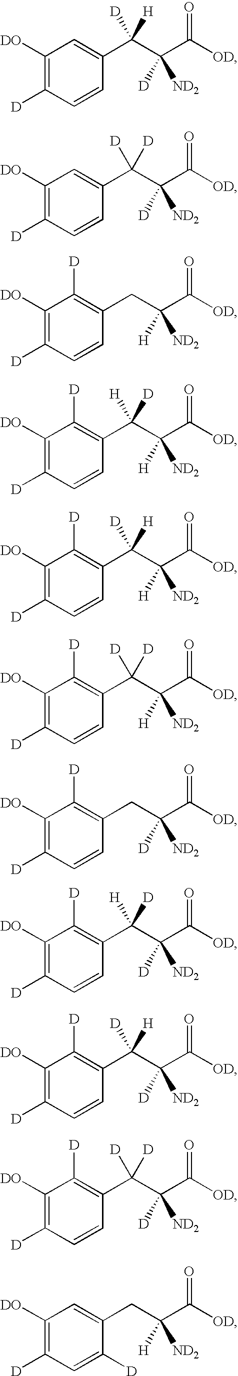

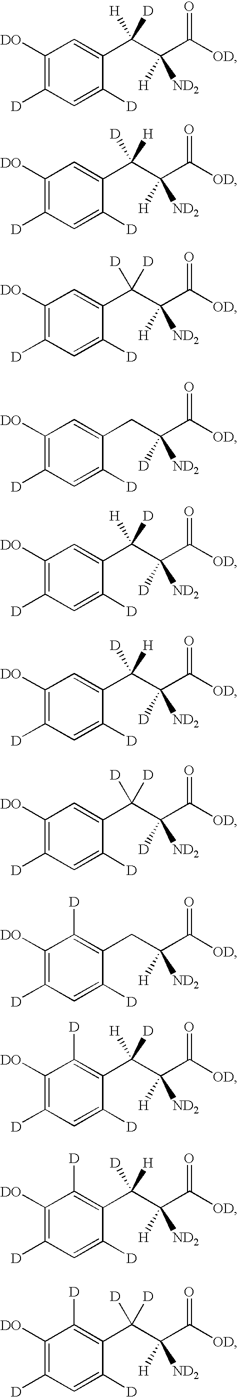

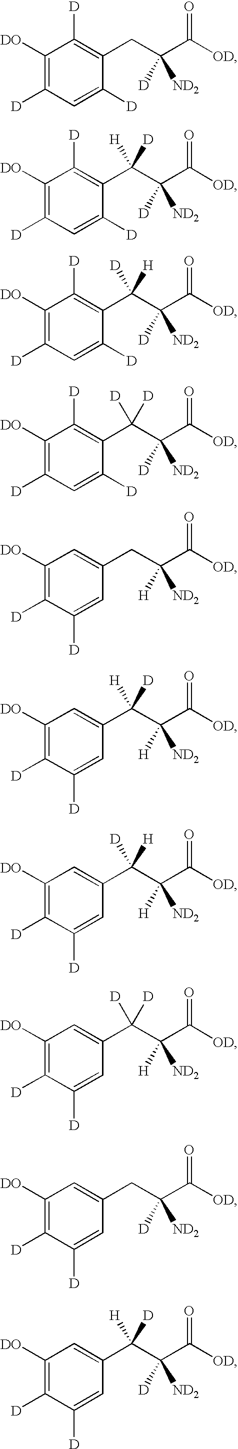

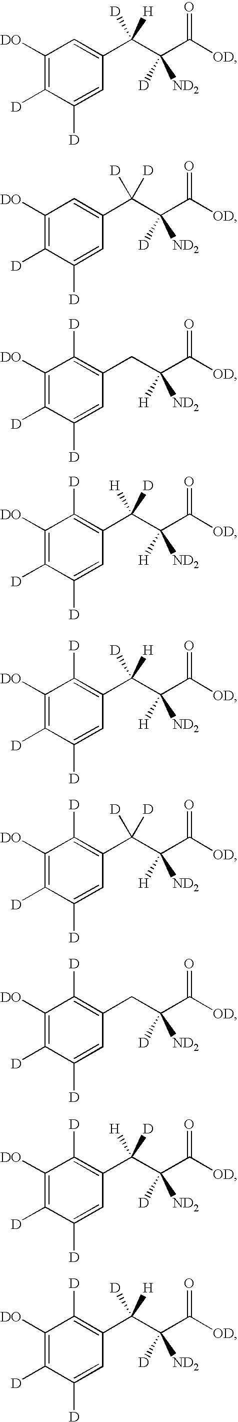

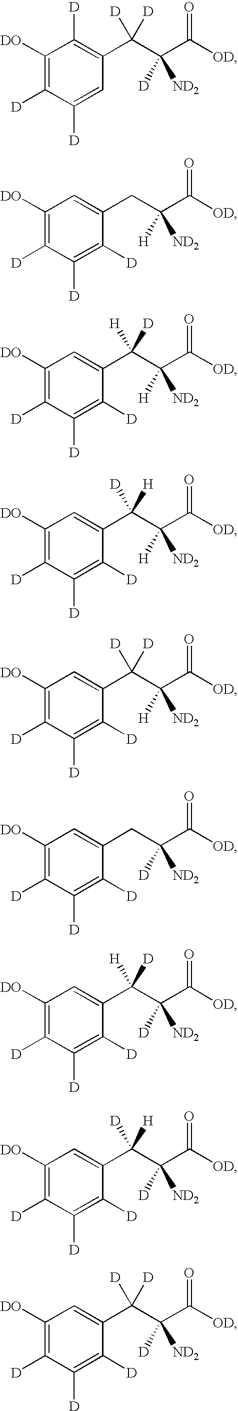

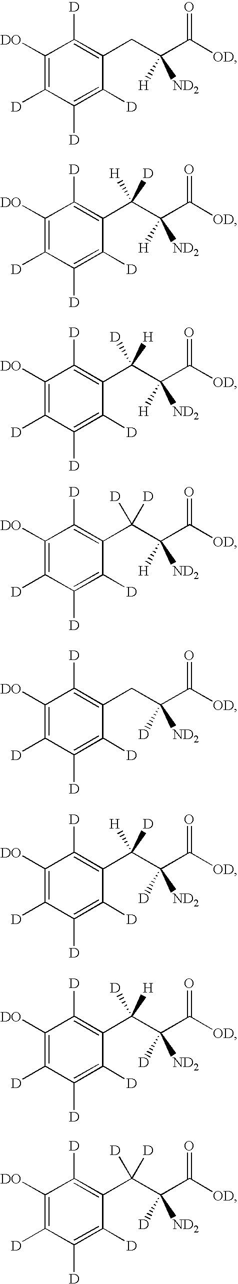

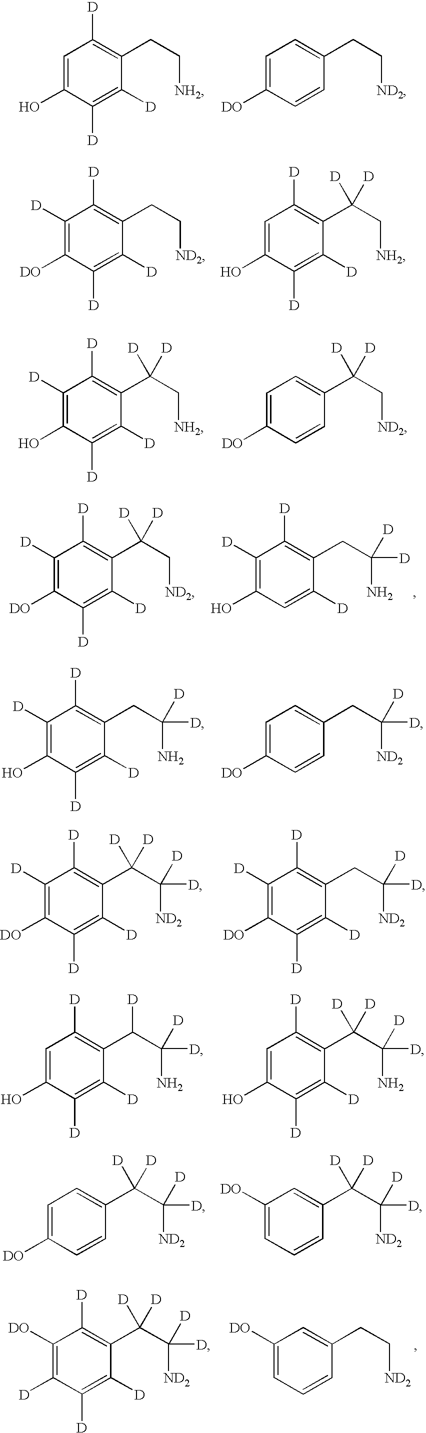

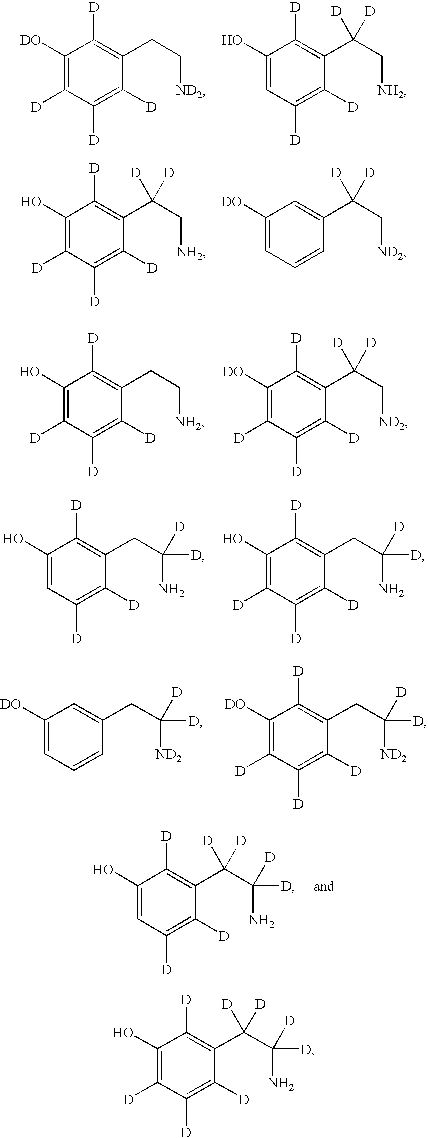

or a pharmaceutically acceptable salt, solvate, or prodrug thereof.

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

### In Vitro Liver Microsomal Stability Assay

Liver microsomal stability assays were conducted at 0.5 mg per mL liver microsome protein with an NADPH-generating system in 2% sodium bicarbonate (2.2 mM NADPH, 25.6 mM glucose 6-phosphate, 6 units per mL glucose 6-phosphate dehydrogenase and 3.3 mM magnesium chloride). Test compounds were prepared as solutions in 20% acetonitrile-water and were added to the assay mixture (final assay concentration 5 microgram per mL) and incubated at 37° C. Final concentration of acetonitrile in the assay should be <1%. Aliquots (50 μL) were taken out at times 0, 7.5, 15, 22.5, and 30 minutes, and diluted with ice cold acetonitrile (200 μL) to stop the reactions. Samples were centrifuged at 12,000 RPM for 10 minutes to precipitate proteins. Supernatants were transferred to microcentrifuge tubes and stored for LC/MS/MS analysis of the degradation half-life of the test compounds. It has thus been found that certain deuterium-enriched compounds disclosed herein, that have been tested in this assay, Examples 2 and 3, showed a decrease in degradation half-life as compared to the non-isotopically enriched drug.

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 minutes. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 minutes. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler et al., Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM sodium phosphate buffer, pH 7.2, containing 0.2% Triton X-100 (monoamine oxidase assay buffer), plus 1 mM kynuramine, and the desired amount of enzyme in 1 mL total volume.

### Monoamine Oxidase B Inhibition and Oxidative Turnover

The procedure is carried out as described in Uebelhack et al., Pharmacopsychiatry 1998, 31(5), 187-192, which is hereby incorporated by reference in its entirety.

### Analysis of Tyrosine and Deuterium Labelled Tyrosine in Tissues and Body Fluids

The procedure is carried out as described in Birgitta Sjöquist, Biomedical Spectrometry 1979, 6(9), 392-395, which is hereby incorporated by reference in its entirety.

### Isotope Dilution GC-MS Analysis of Tyrosine Oxidation Products in Proteins and Tissues

The procedure is carried out as described in Heinecke J. W., Methods in Biological Oxidative Stress 2003, 93-100, which is hereby incorporated by reference in its entirety.

Determination of m-Tyrosine in Human Plasma by HPLC

The procedure is carried out as described in Ishimitsu et al., Chemical & Pharmaceutical Bulletin 1982, 30(5), 1889-91, which is hereby incorporated by reference in its entirety.

### Detecting L-Dopa and Dopamine in Rat Plasma Using Electrospray LC/MS/MS

The procedure is carried out as described in Li et al., Journal of Pharmaceutical and Biomedical Analysis 2000, 24(2), 325-333, which is hereby incorporated by reference in its entirety.

### Tyrosine Hydroxylase Assay Using HPLC to Quantify of L-dopa and L-tyrosine

The procedure is carried out as described in Olsovska et al, Biomedical Chromatography 2007, 21(12), 1252-1258, which is hereby incorporated by reference in its entirety.

### Determination of Tyrosine Metabolites by GC-Negative-Ion Chemical-Ionization MS

The procedure is carried out as described in Shimamura et al., Journal of Chromatography 1986, 374(1), 17-26, which is hereby incorporated by reference in its entirety.

### Tyrosine Hydroxylase Assay for Detection of Low Levels of Enzyme Activity in Peripheral Tissues

The procedure is carried out as described in Hooper et al., Journal of Chromatography, B: Biomedical Sciences and Applications 1997, 694(2), 317-324, which is hereby incorporated by reference in its entirety.

### Assays for Tyrosine Hydroxylase and Dopa Oxidase Activities of Tyrosinase

The procedure is carried out as described in Winder et al., European Journal of Biochemistry 1991, 198(2), 317-26, which is hereby incorporated by reference in its entirety.

### HPLC-Based Tyramine Assay

The procedure is carried out as described in Scaro et al., Journal of Liquid Chromatography 1980, 3(4), 537-43, which is hereby incorporated by reference in its entirety.

From the foregoing description, one skilled in the art can ascertain the essential characteristics of this invention, and without departing from the spirit and scope thereof, can make various changes and modifications of the invention to adapt it to various usages and conditions.

What is claimed is:

1. A compound having structural Formula I 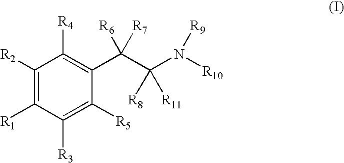
or a pharmaceutically acceptable salt thereof, wherein:
R₁ and R₂ are independently selected from the group consisting of hydrogen, deuterium, —OH, and —OD, wherein at least one of R₁ and R₂ is hydrogen or deuterium;
R₃-R₁₀ are independently selected from the group consisting of hydrogen and deuterium;
R₁₁ is selected from the group consisting of hydrogen, deuterium, —CO₂H, —CO₂D, and —CO₂R₁₂, wherein R₁₂ is alkyl or deuterated alkyl;
at least one of R₁-R₁₂ is deuterium or contains deuterium; and
with the proviso that the compound cannot be selected from the group consisting of:

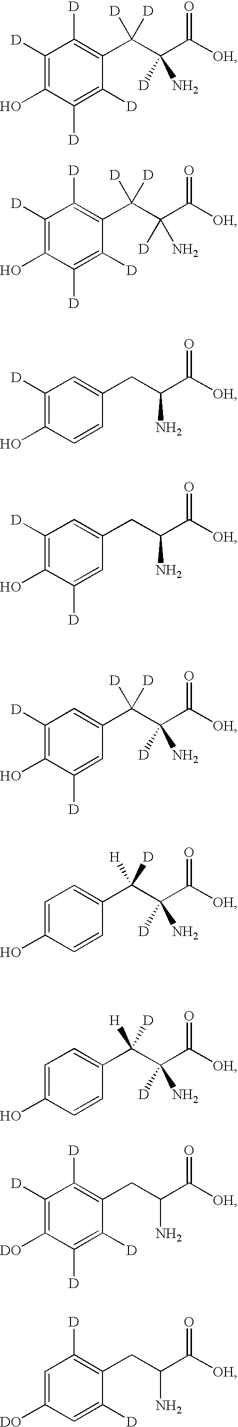

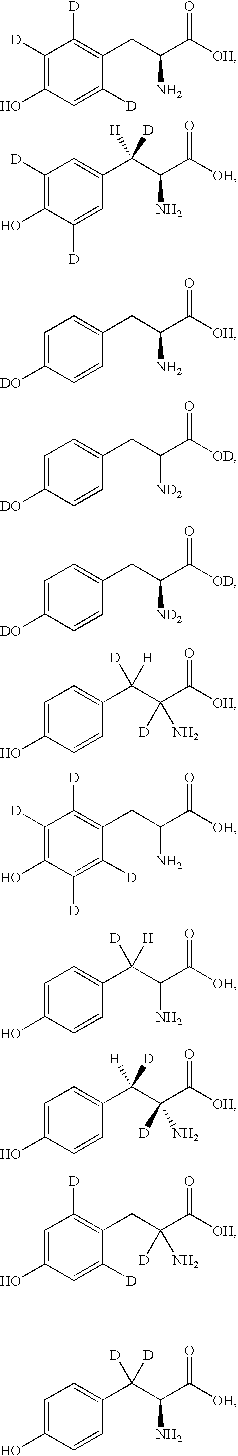

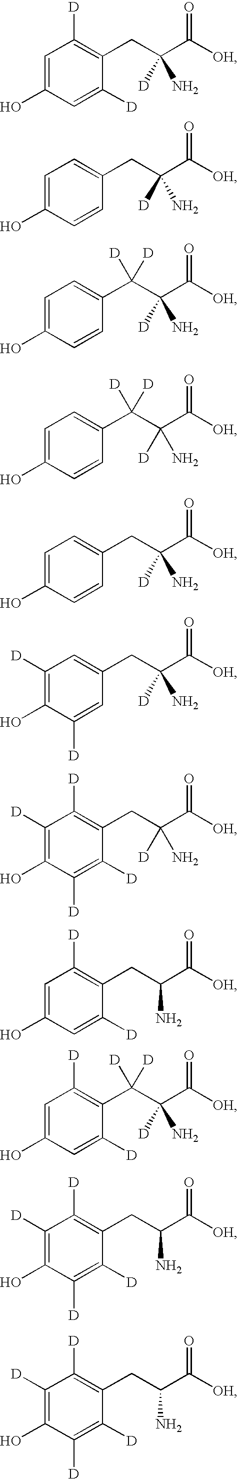

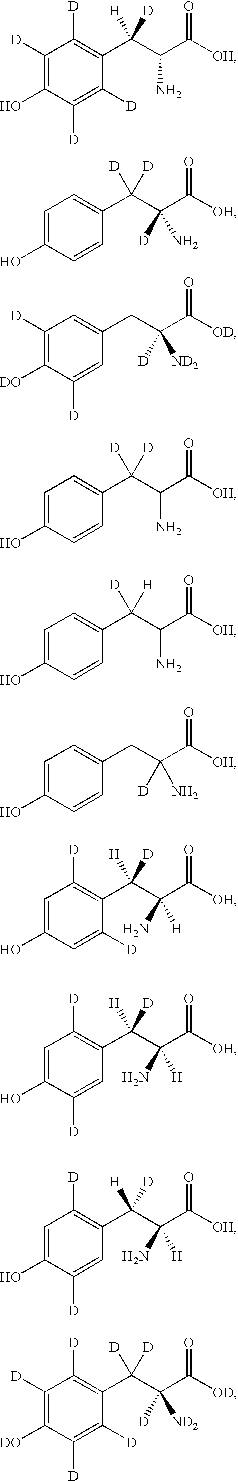

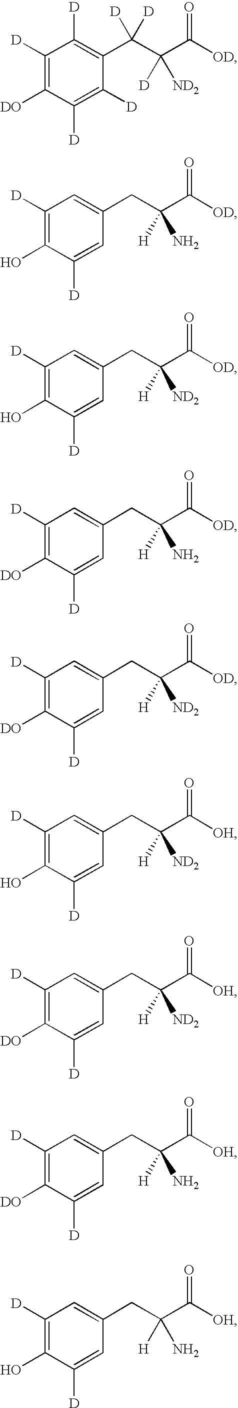

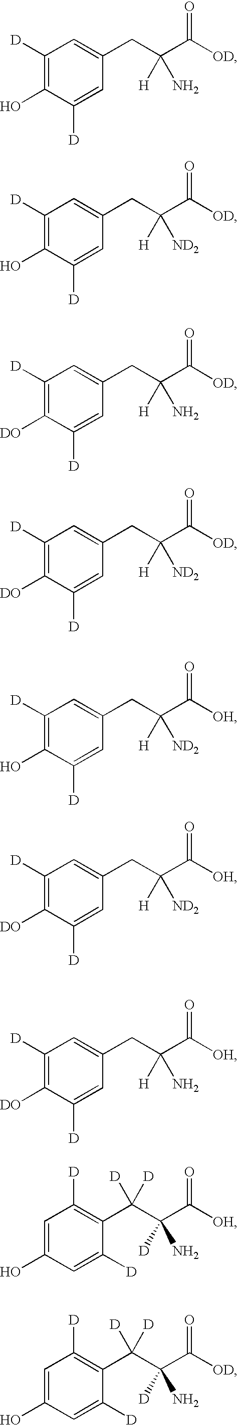

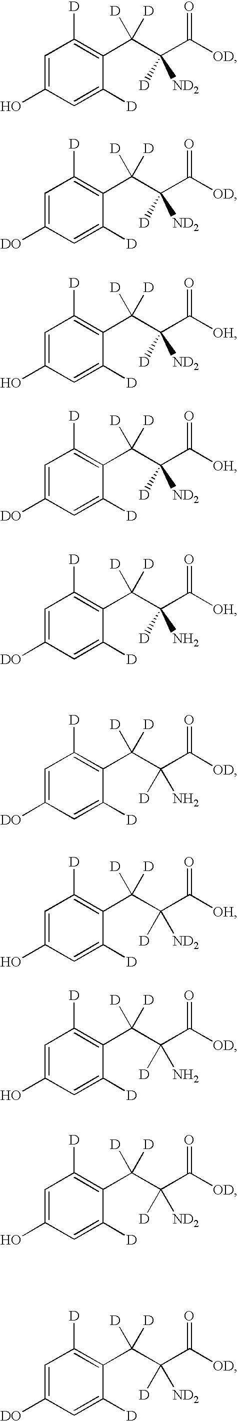

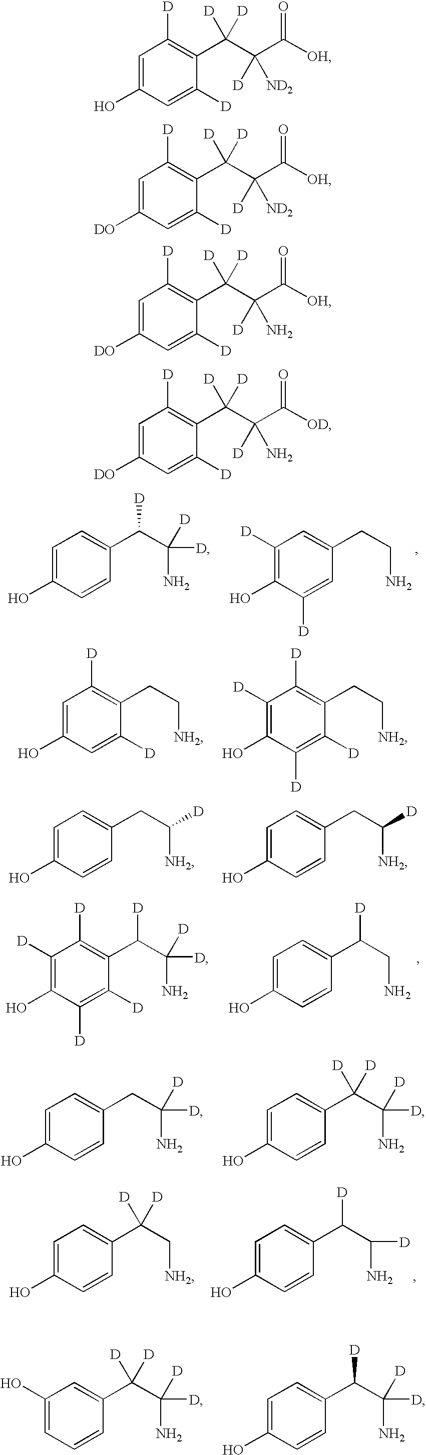

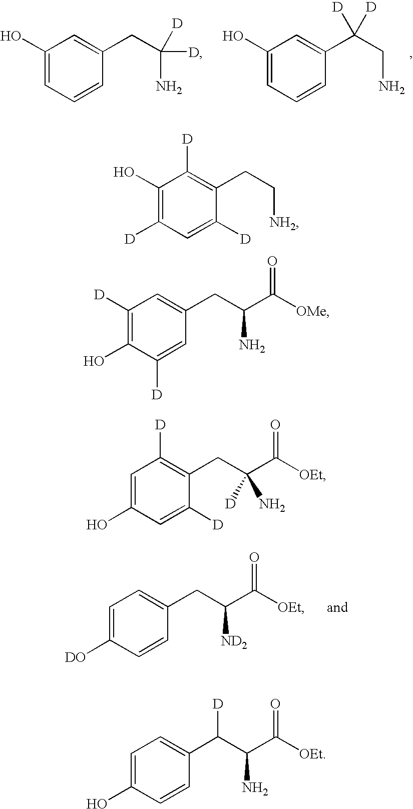

2. The compound as recited in claim 1 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

3. The compound as recited in claim 1 wherein at least one of R₁-R₁₂ independently has deuterium enrichment of no less than about 10%.

4. The compound as recited in claim 1 wherein at least one of R₁-R₁₂ independently has deuterium enrichment of no less than about 50%.

5. The compound as recited in claim 1 wherein at least one of R₁-R₁₂ independently has deuterium enrichment of no less than about 90%.

6. The compound as recited in claim 1 wherein at least one of R₁-R₁₂ independently has deuterium enrichment of no less than about 98%.

7. The compound as recited in claim 1 wherein said compound has a structural formula selected from the group consisting of: 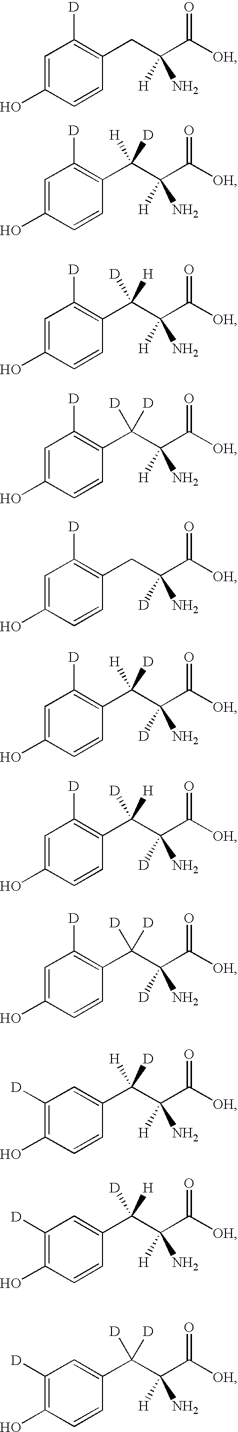 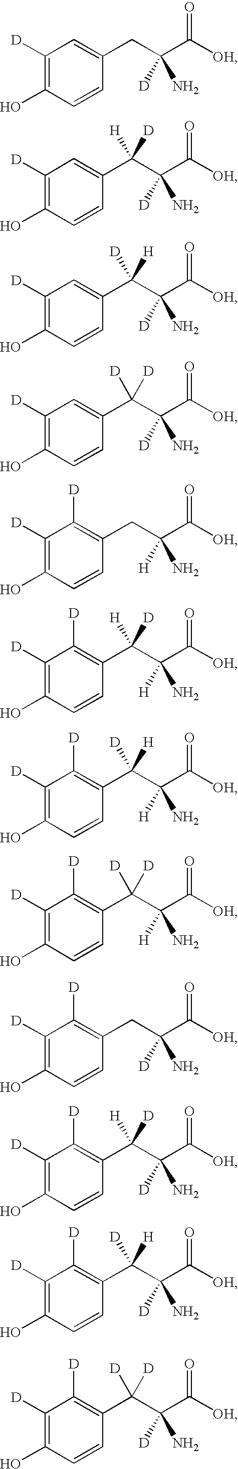 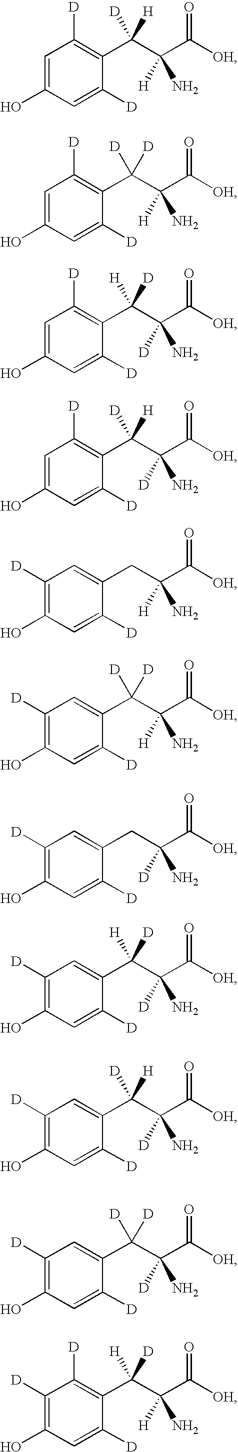 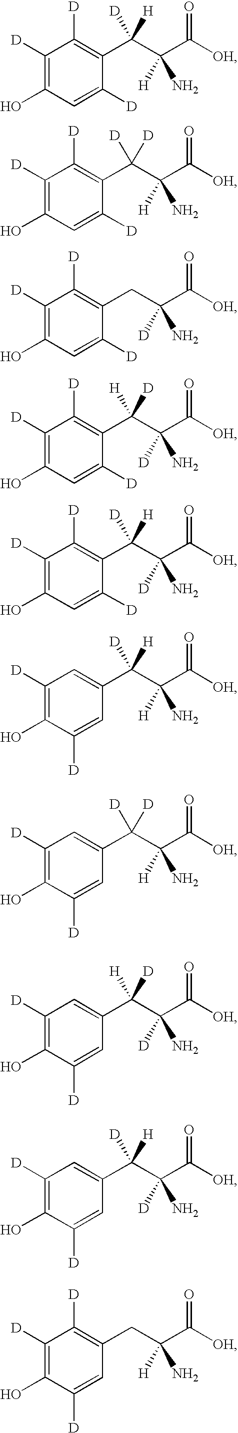 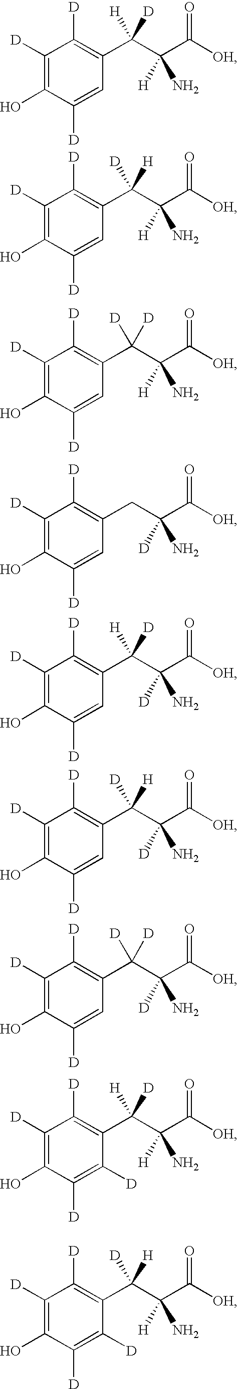 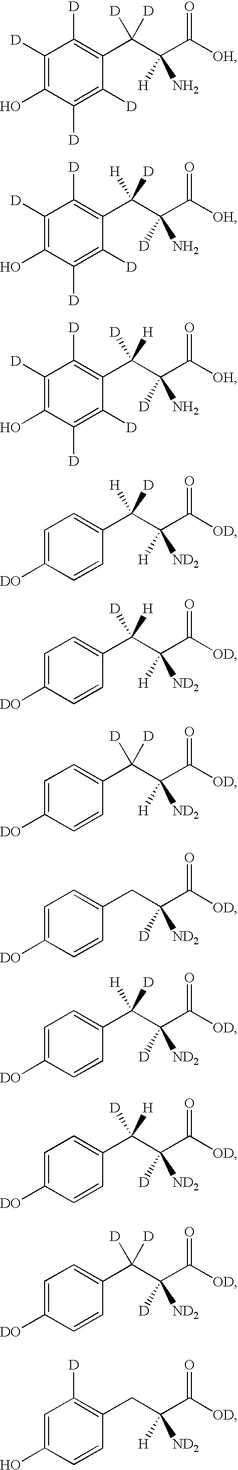 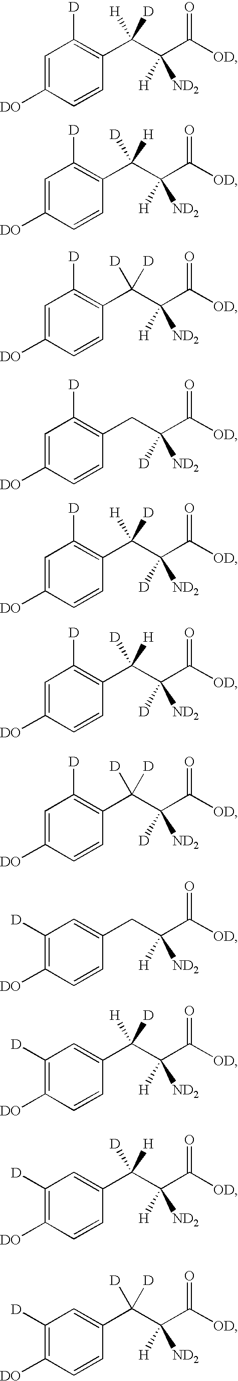 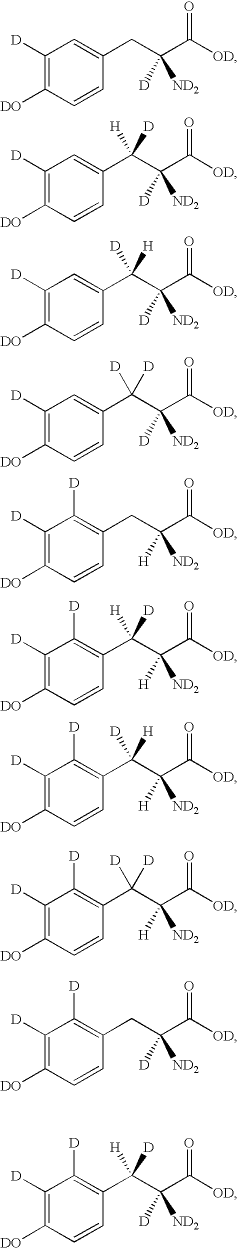 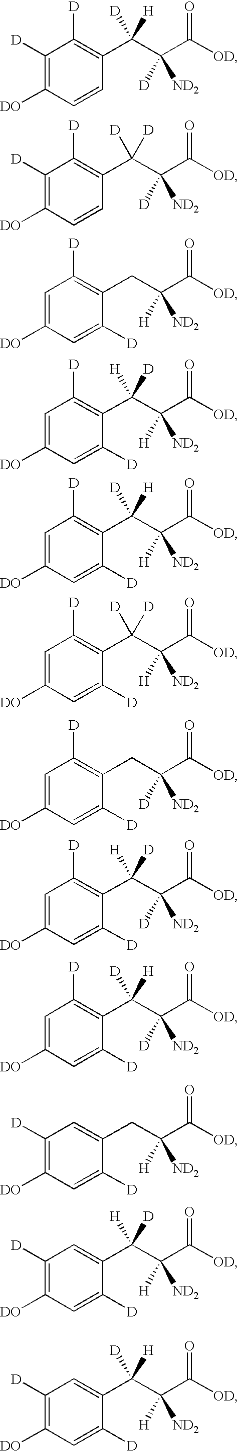 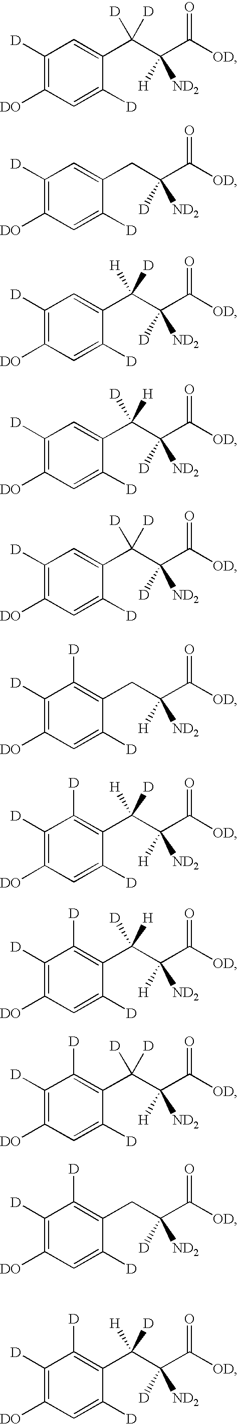 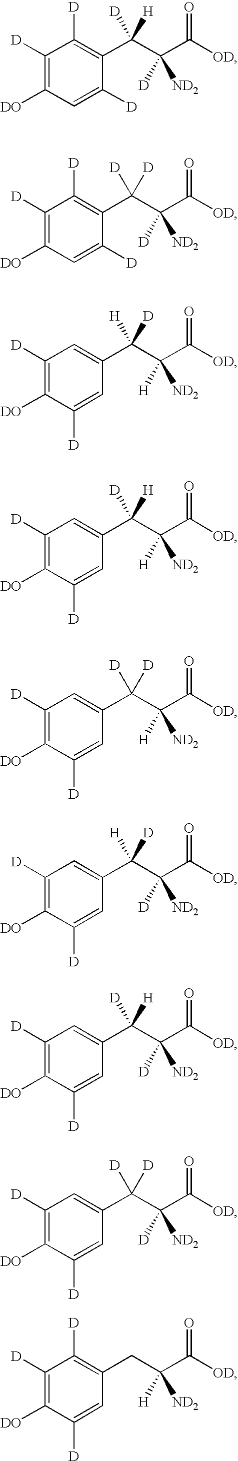 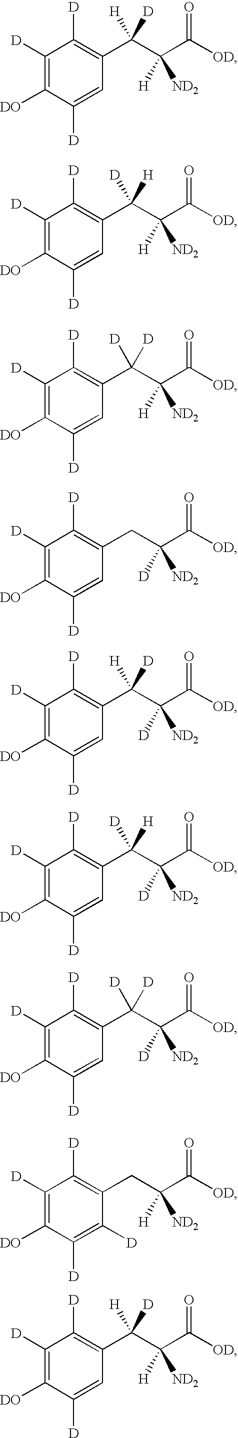 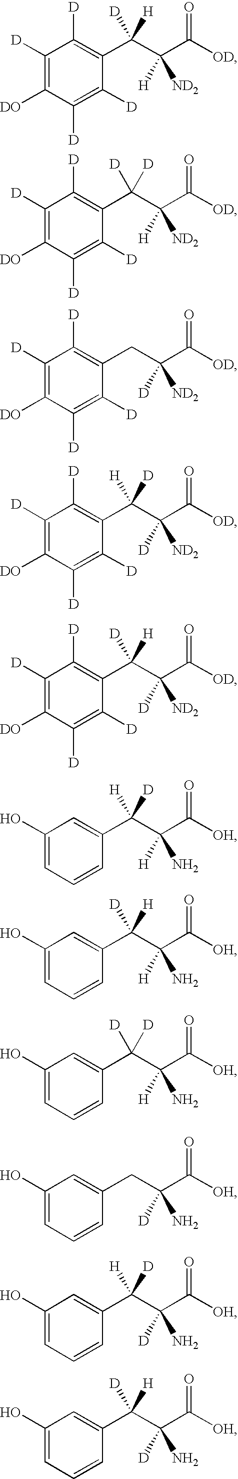 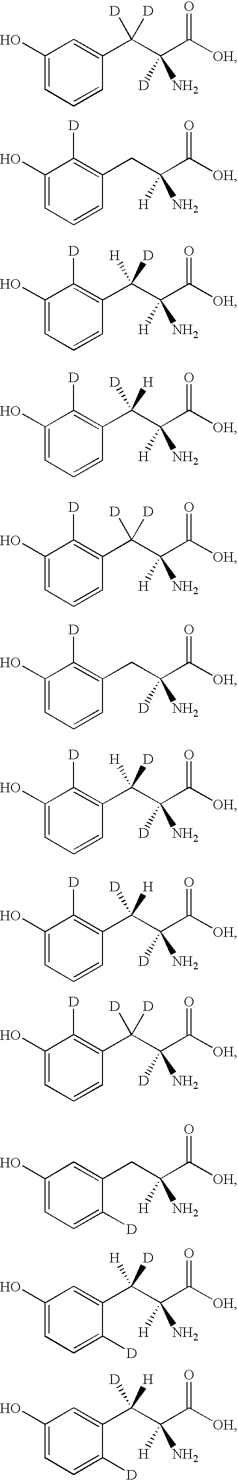 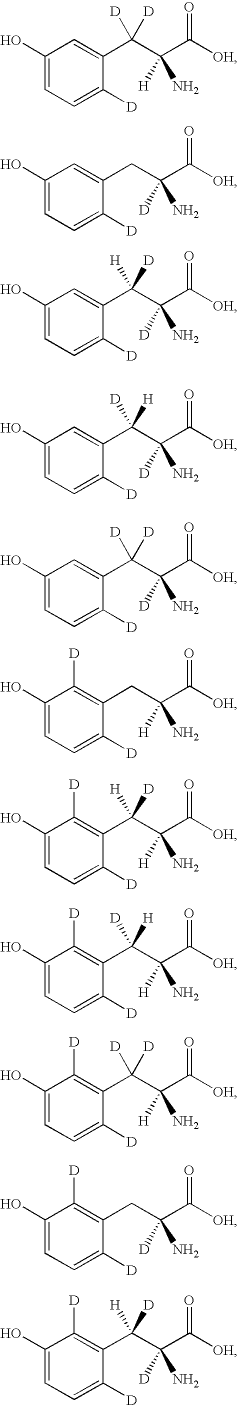 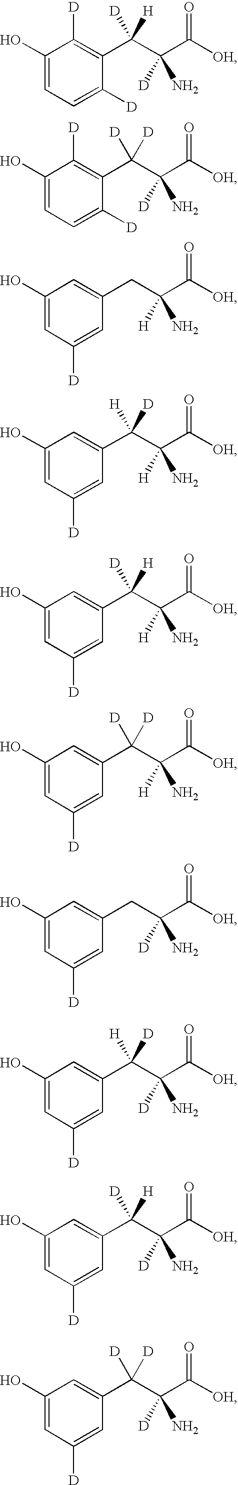 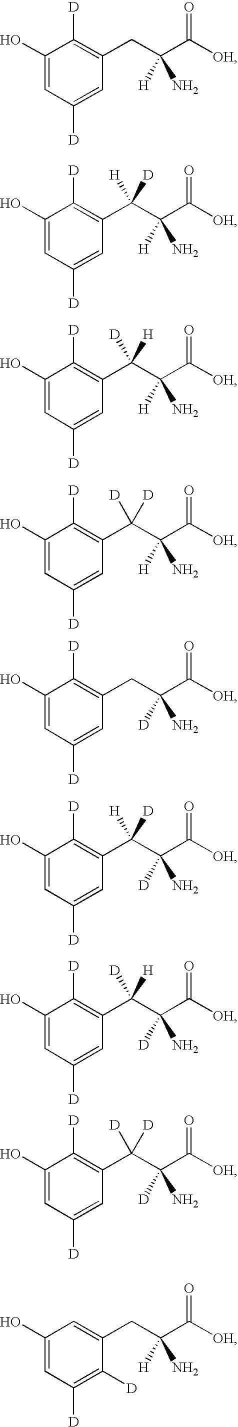 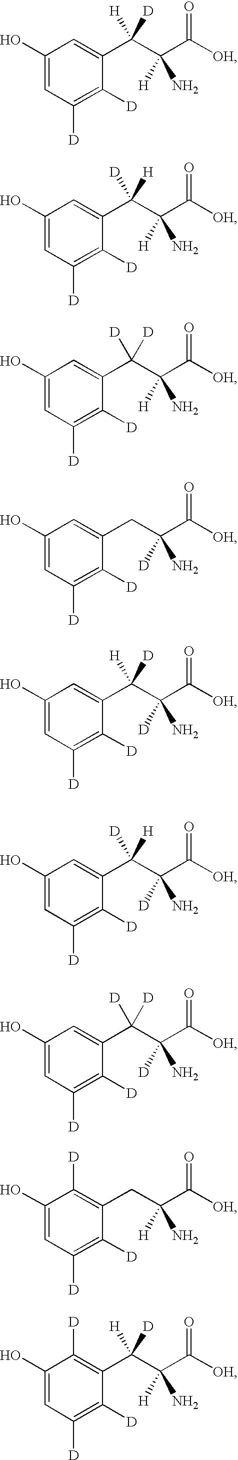 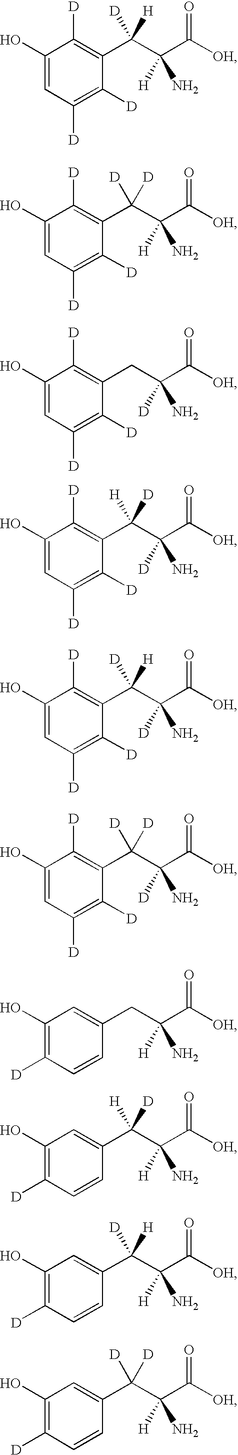 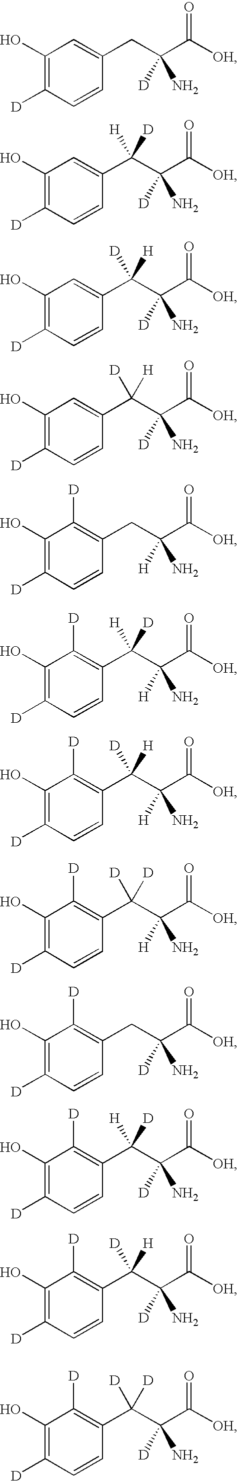 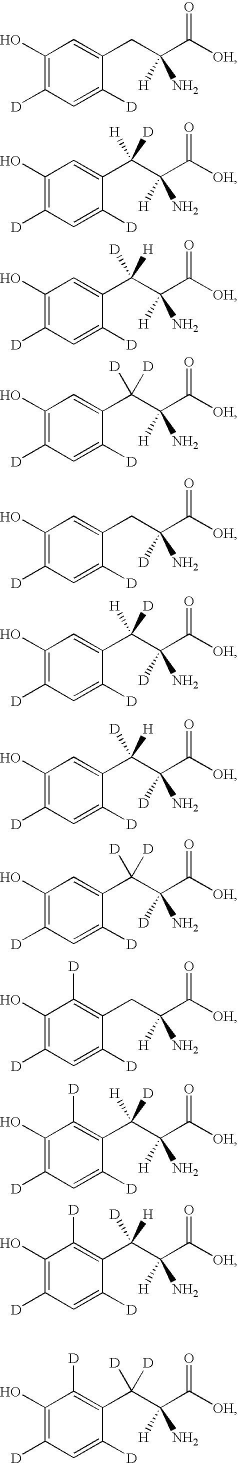 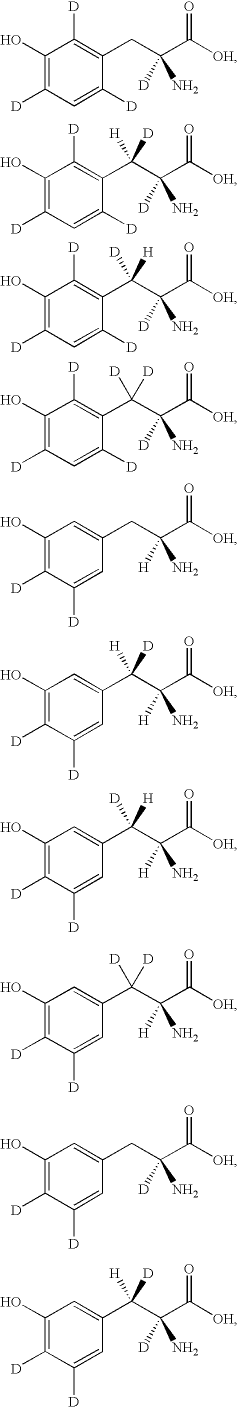 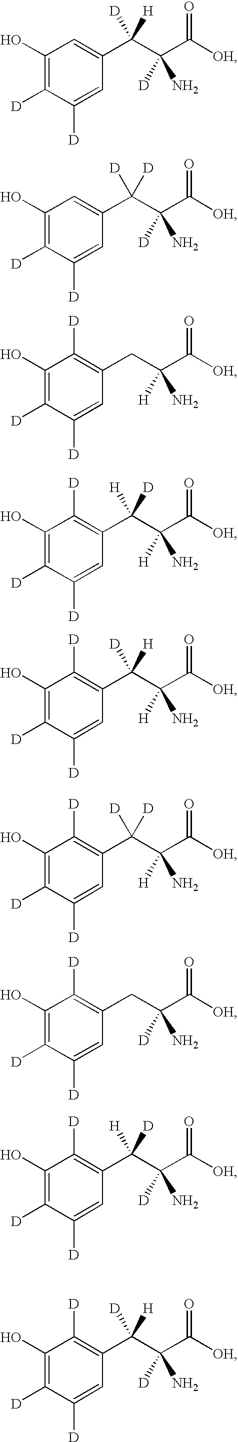 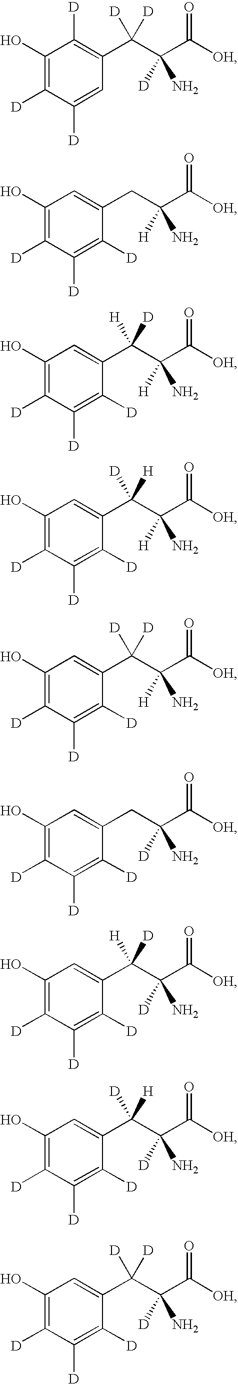 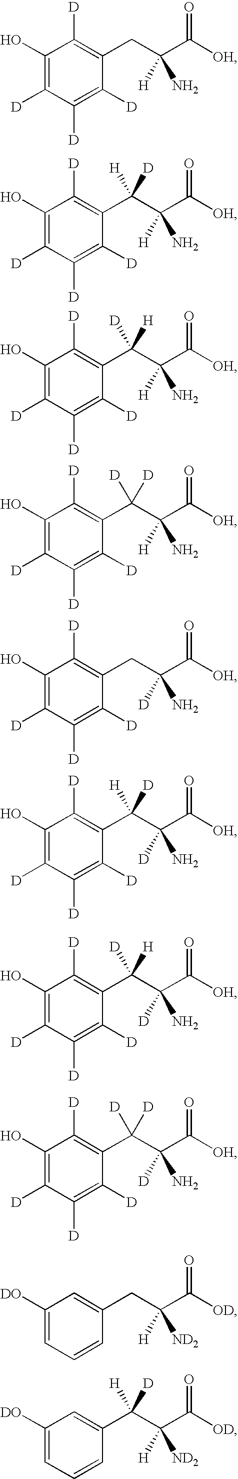 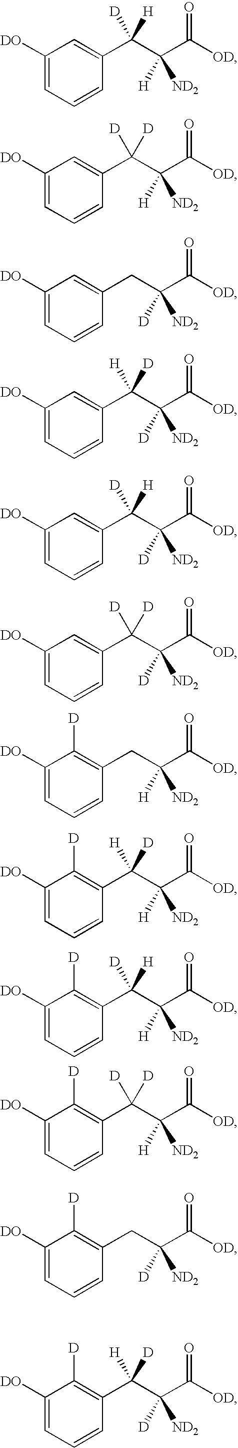 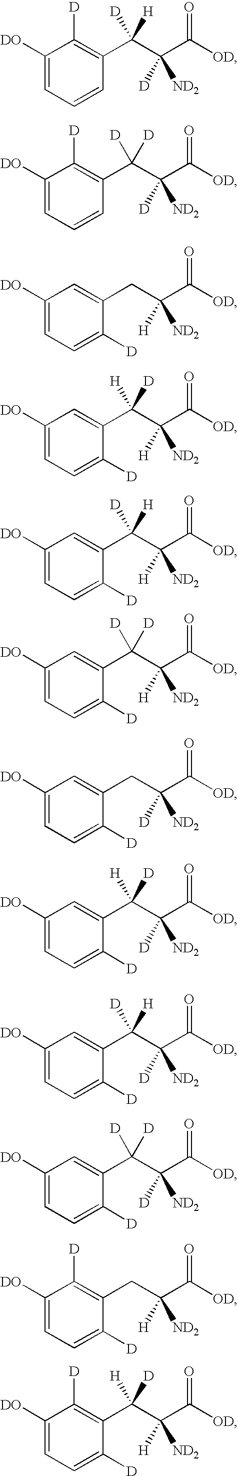 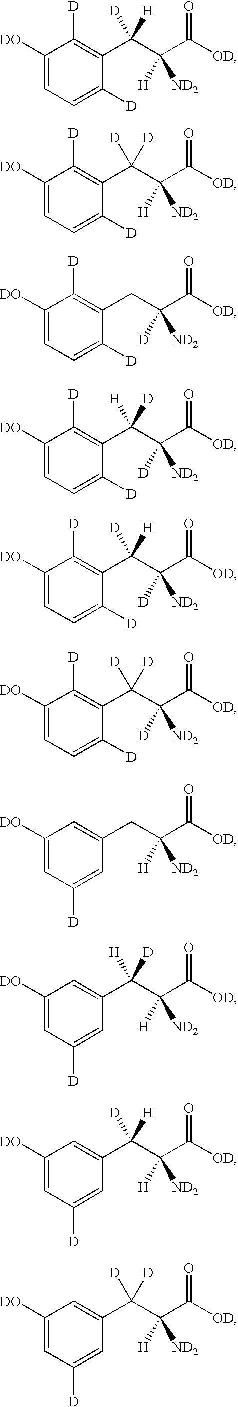 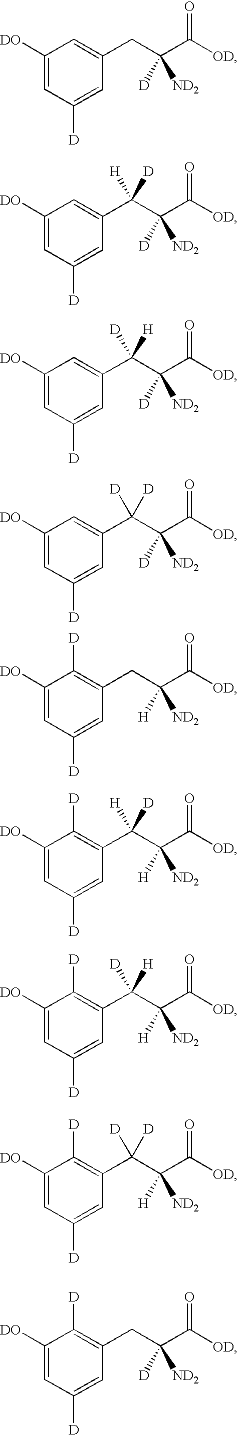 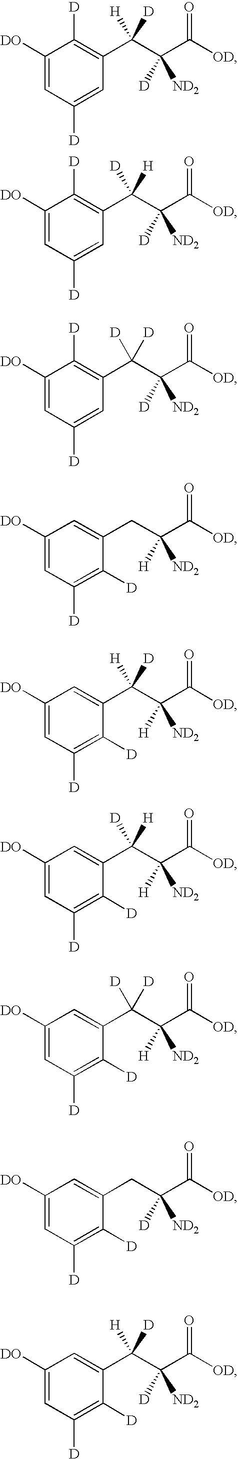 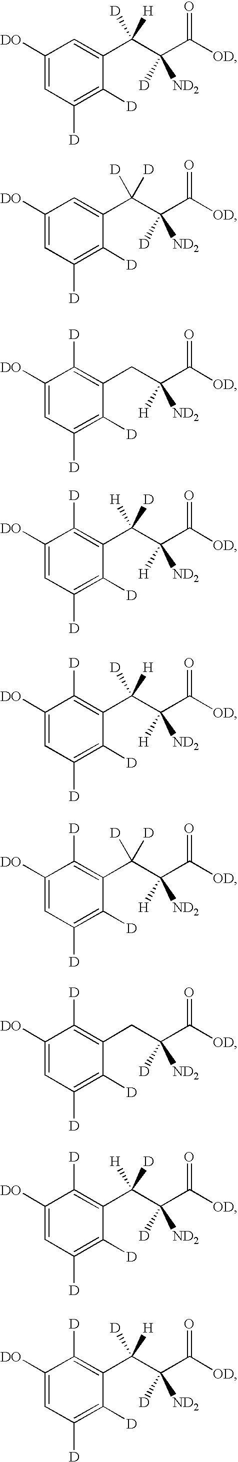 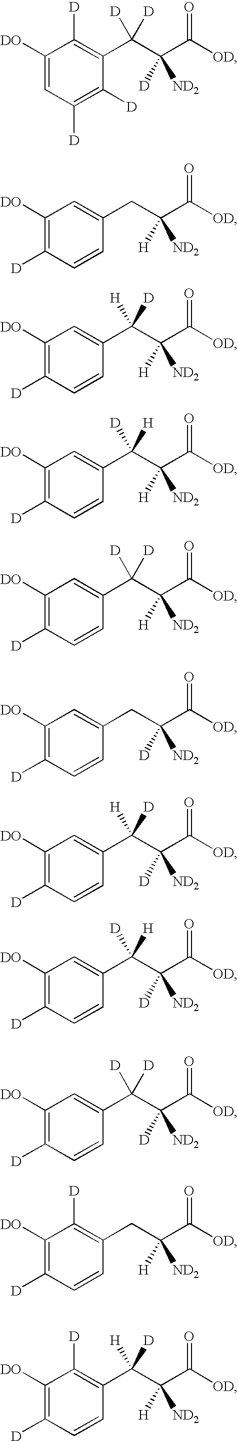 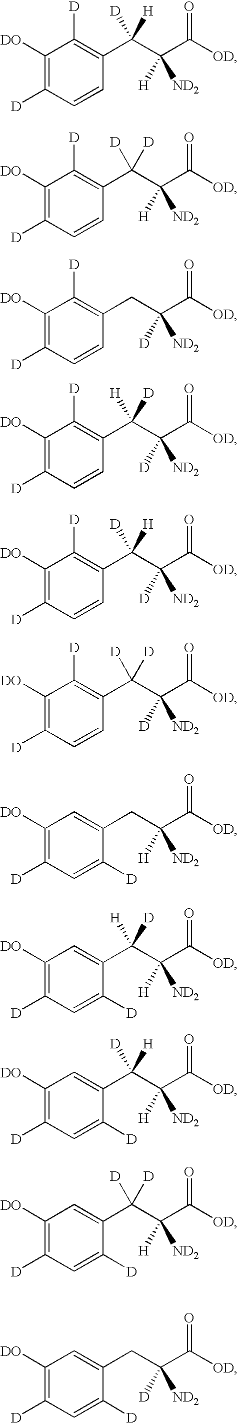 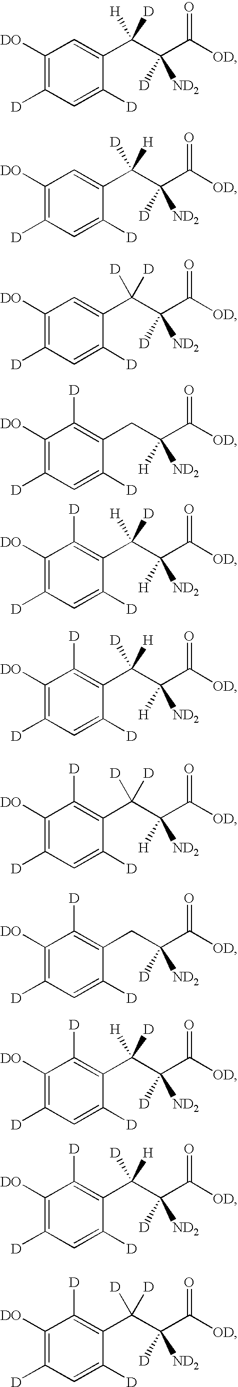 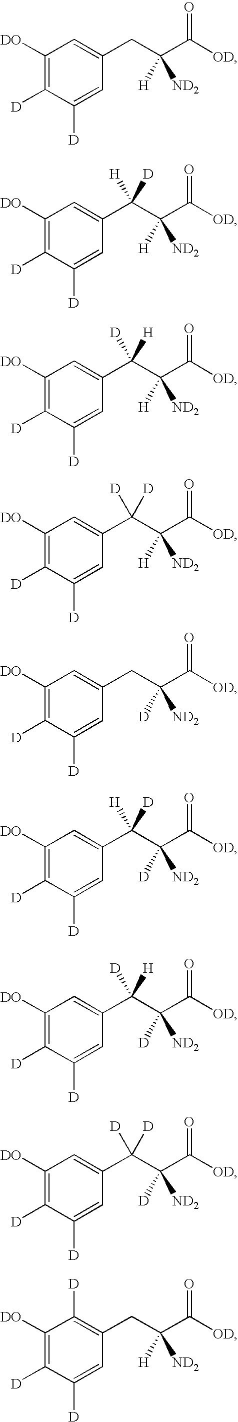 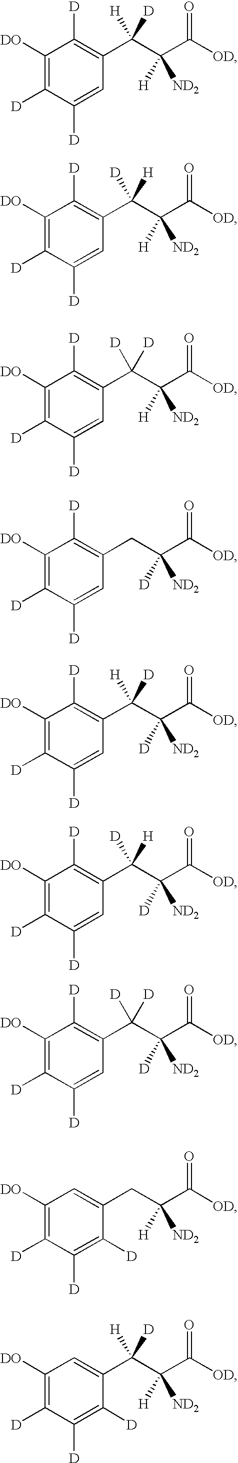 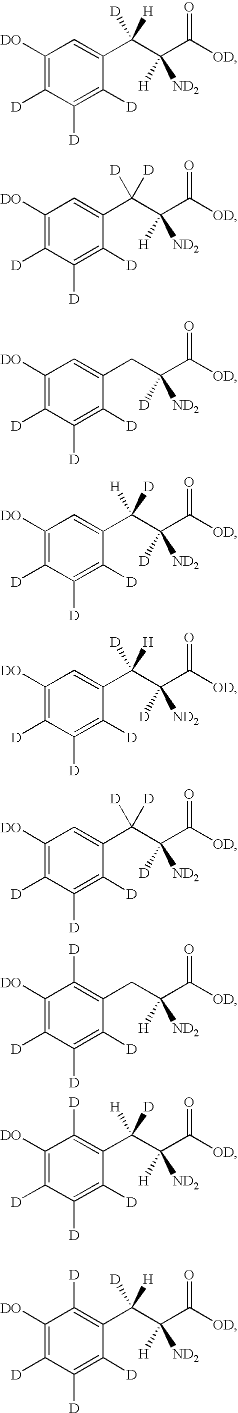 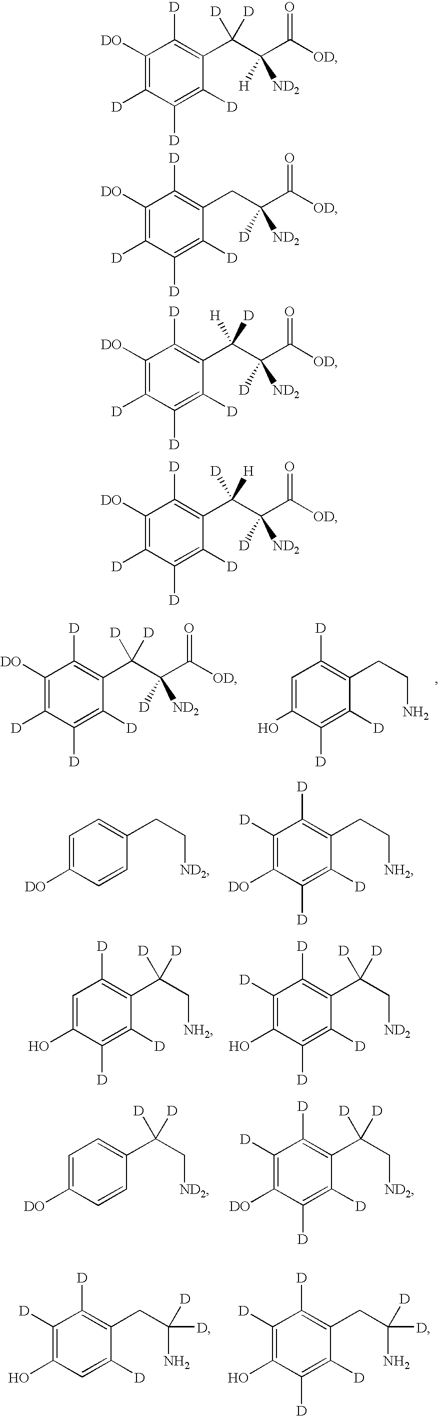 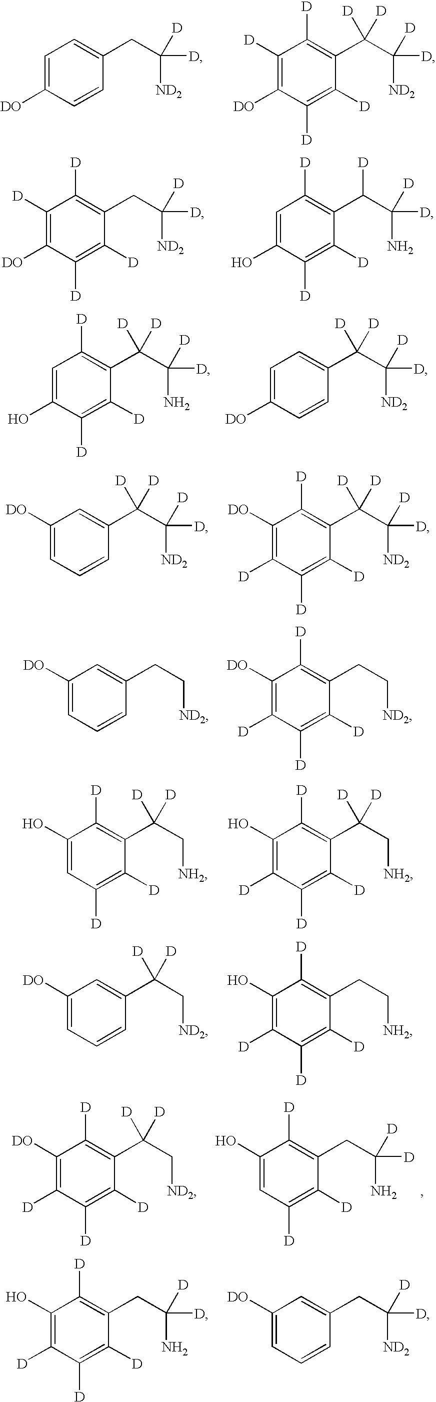 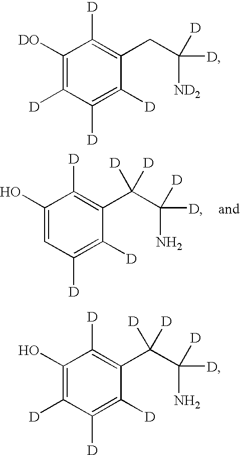
or a pharmaceutically acceptable salt thereof.

8. The compound as recited in claim 7 wherein each position represented as D has deuterium enrichment of no less than about 10%.

9. The compound as recited in claim 7 wherein each position represented as D has deuterium enrichment of no less than about 50%.

10. The compound as recited in claim 7 wherein each position represented as D has deuterium enrichment of no less than about 90%.

11. The compound as recited in claim 7 wherein each position represented as D has deuterium enrichment of no less than about 98%.

12. The compound as recited in claim 1 wherein said compound has a structural formula selected from the group consisting of: 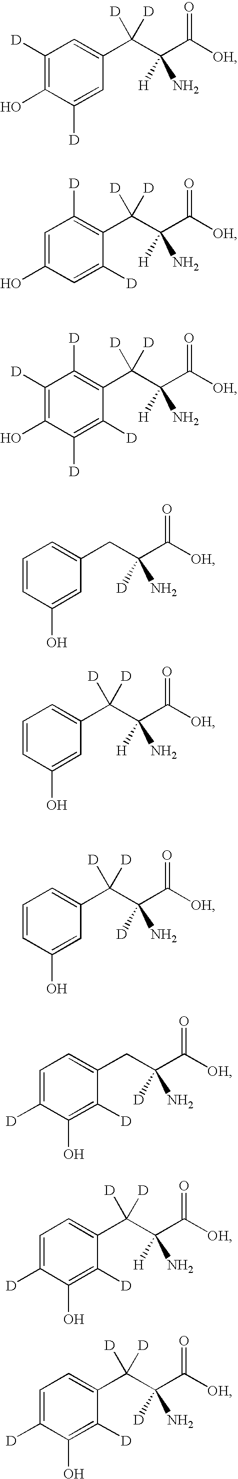 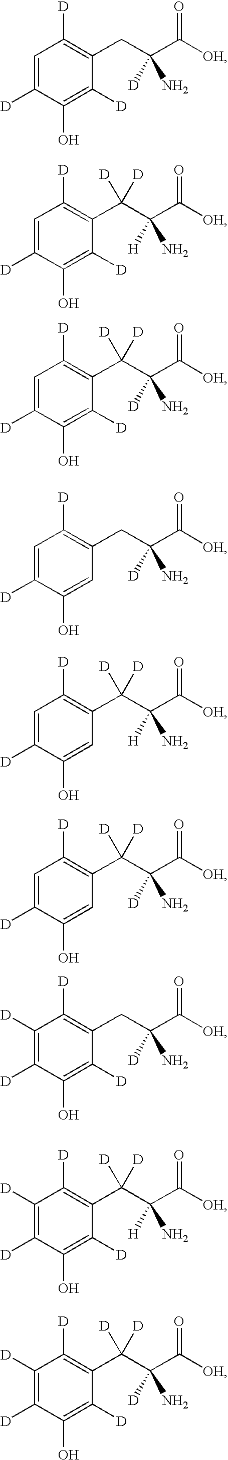 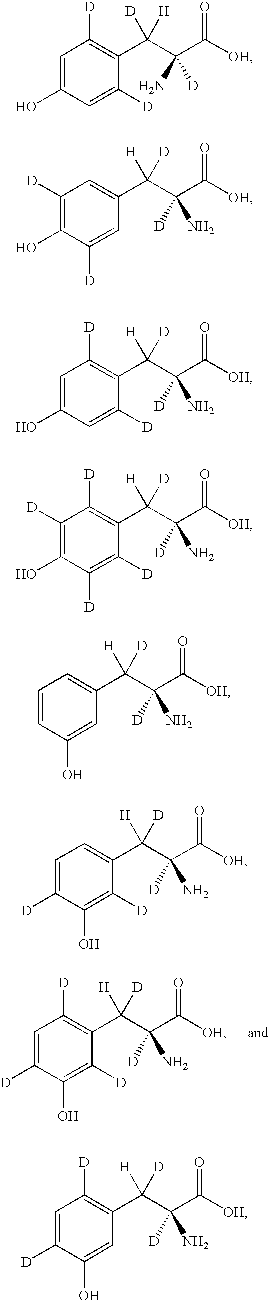
or a pharmaceutically acceptable salt thereof.

13. The compound as recited in claim 12 wherein said compound has the structural formula:

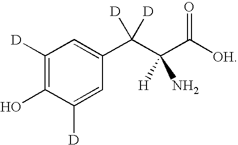

14. The compound as recited in claim 12 wherein said compound has the structural formula:

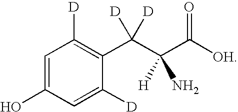

15. The compound as recited in claim 12 wherein said compound has the structural formula:

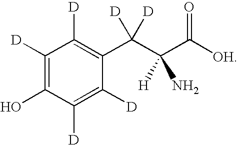

16. The compound as recited in claim 12 wherein said compound has the structural formula:

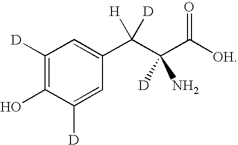

17. The compound as recited in claim 12 wherein said compound has the structural formula:

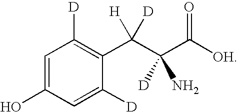

18. The compound as recited in claim 12 wherein said compound has the structural formula:

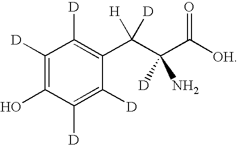

19. The compound as recited in claim 12 wherein said compound has the structural formula:

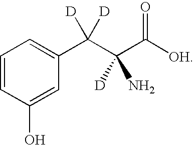

20. The compound as recited in claim 12 wherein said compound has the structural formula:

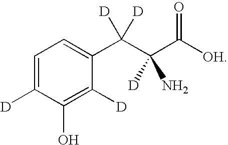

21. The compound as recited in claim 12 wherein said compound has the structural formula:

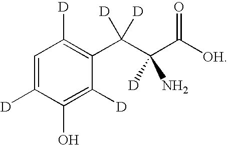

22. The compound as recited in claim 12 wherein said compound has the structural formula:

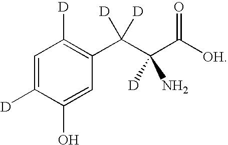

23. The compound as recited in claim 12 wherein said compound has the structural formula:

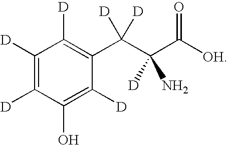

24. The compound as recited in claim 12 wherein said compound has the structural formula:

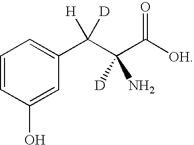

25. The compound as recited in claim 12 wherein said compound has the structural formula:

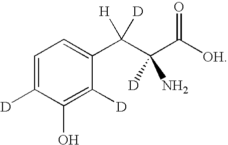

26. The compound as recited in claim 12 wherein said compound has the structural formula:

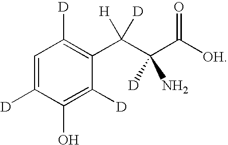

27. The compound as recited in claim 12 wherein said compound has the structural formula:

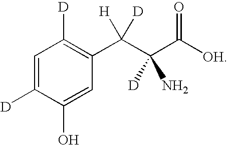

28. A pharmaceutical composition comprising a pharmaceutically acceptable carrier together with a compound having structural Formula I: 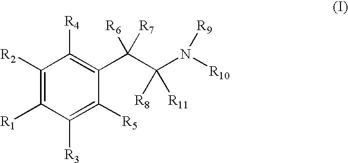
or a pharmaceutically acceptable salt thereof, wherein:
R₁ and R₂ are independently selected from the group consisting of hydrogen, deuterium, —OH, and —OD, wherein at least one of R₁ or R₂ is hydrogen or deuterium;
R₃-R₁₀ are independently selected from the group consisting of hydrogen and deuterium;
R₁₁ is selected from the group consisting of hydrogen, deuterium, CO₂H, —CO₂D, and —CO₂R₁₂, wherein R₁₂ is an alkyl, or deuterated alkyl; and
at least one of R₁-R₁₂ is deuterium or contains deuterium.

29. A method of treatment of an hormone-mediated disorder or a pigment-mediated disorder comprising the administration of a therapeutically effective amount of a compound having structural Formula I: 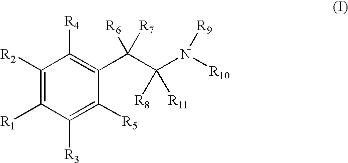
or a pharmaceutically acceptable salt thereof, wherein:
R₁ and R₂ are independently selected from the group consisting of hydrogen, deuterium, —OH, and —OD, wherein at least one of R₁ or R₂ is hydrogen or deuterium;
R₃-R₁₀ are independently selected from the group consisting of hydrogen and deuterium;
R₁₁ is selected from the group consisting of hydrogen, deuterium, CO₂H, —CO₂D, and —CO₂R₁₂, wherein R₁₂ is an alkyl, or deuterated alkyl; and
at least one of R₁-R₁₂ is deuterium or contains deuterium.

30. The method as recited in claim 29 wherein the hormone-mediated disorder or pigment-mediated disorder is selected from the group consisting of stress-associated conditions, obesity, alcohol withdrawal syndrome, drug dependence, depression, Parkinson's disease, narcolepsy, Alzheimer's disease, phenylketonuria, multi-infarct dementia, vitiglio, chronic uremia, HIV infection of the central nervous system, AIDS dementia, amyotrophic lateral sclerosis, hereditary hemorrhage with amyloidosis-Dutch type, cerebral amyloid angiopathy, Down's syndrome, spongiform encephalopathy, Creutzfeldt-Jakob disease, hemorrhagic shock, restless leg syndrome, dystonia, carbon monoxide poisoning, cyanide poisoning, methanol poisoning, and manganese poisoning.

31. The method as recited in claim 29 further comprising the administration of an additional therapeutic agent.

32. The method as recited in claim 31 wherein said additional therapeutic agent is selected from the group consisting of dietary supplements, dopamine agonists, monoamine oxidase inhibitors, dopamine prodrugs, L-dopa metabolism suppressors, adamantine-based agents, SNRIs, SSRIs, acetylcholinesterase inhibitors, TCAs, barbituates, benzodiazepines, amphetamine-like stimulants, platelet aggregation inhibitors, statins, anticoagulants, thrombolytics, fibrates, bile acid sequestrants, CETP inhibitors, lipid modifying agents, NSAIDs, anti-bacterial agents, anti-fungal agents, sepsis treatments, steroidals, local or general anesthetics, NRIs, DARIs, SNRIs, sedatives, NDRIs, SNDRIs, monoamine oxidase inhibitors, hypothalamic phospholipids, ECE inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIIa Inhibitors and Factor Xa Inhibitors, renin inhibitors, NEP inhibitors, vasopepsidase inhibitors, squalene synthetase inhibitors, anti-atherosclerotic agents, MTP Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, antiinflammatories, antiproliferatives, chemotherapeutic agents, immunosuppressants, anticancer agents and cytotoxic agents, antimetabolites, antibiotics, farnesyl-protein transferase inhibitors, hormonal agents, microtubule-disruptor agents, microtubule-stablizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

33. The method as recited in claim 31 wherein said dietary supplement is selected from the group consisting of ferrous iron, tetrahydrofolic acid, pyridoxal phosphate, NADH, pyridoxine, nicotinamide, vitamin C, vitamin E, vitamin B12, vitamin B3, curcumin, folic acid, Coenzyme Q10, Mucuna pruriens extract, and MitoQ.

34. The method as recited in claim 31 wherein said dopamine agonist is selected from the group consisting of A-412,997, apomorphine, bromocriptine, cabergoline, dihydrexidine, dihydroergocryptine mesylate, fenoldopam, lisuride, pergolide, piribedil, pramipexole, propylnorapomorphine, quinpirole, ropinirole, rotigotine, SKF 38393, and SKF 82958.

35. The method as recited in claim 31 wherein said monoamine oxidase inhibitor is selected from the group consisting of iproclozide, iproniazid, isocarboxazid, nialamide, pargyline, phenelzine, rasagiline, selegiline, toloxatone, tranylcypromine, brofaromine, harmaline, moclobemide, linezolid, and dienolide kavapyrone desmethoxyyangonin.

36. The method as recited in claim 31 wherein said dopamine prodrug is selected from the group consisting of droxidopa, levodopa, melevodopa, and etilevodopa.

37. The method as recited in claim 31 wherein said L-dopa metabolism suppressor is selected from the group consisting of carbidopa, benserazide, tolcapone, and entacapone.

38. The method as recited in claim 31 wherein said adamantine-based agent is selected from the group consisting of amantadine, memantine, and rimantadine.

39. The method as recited in claim 31 wherein said SNRI is selected from the group consisting of bicifadine, desvenlafaxine, duloxetine, milnacipran, nefazodone, and venlafaxine.

40. The method as recited in claim 31 wherein said SSRI is selected from the group consisting of alaproclate, citalopram, dapoxetine, escitalopram, etoperidone, fluoxetine, fluvoxamine, paroxetine, sertraline, and zimelidine.

41. The method as recited in claim 31 wherein said acetylcholinesterase inhibitor is selected from the group consisting of metrifonate, physostigmine, neostigmine, pyridostigmine, ambenonium, demarcarium, rivastigmine, galantamine, donepezil, tacrine, and edrophonium.

42. The method as recited in claim 31 wherein said TCA is selected from the group consisting of clomipramine, nefazodone, trazodone, amitriptyline, amoxapine, butriptyline, desipramine/lofepramine, dibenzepin, dothiepin, doxepin, imipramine, iprindole, melitracen, nortriptyline, opipramol, protriptyline, trimipramine, maprotiline and amineptine.

43. The method as recited in claim 31 wherein said barbiturate is selected from the group consisting of allobarbital, alphenal, amobarbital, aprobarbital, barbexaclone, barbital, brallobarbital, brophebarbital, bucolome, butabarbital, butalbital, butobarbital, butallylonal, crotylbarbital, cyclobarbital, cyclopal, enallylpropymal, ethallobarbital, febarbamate, heptabarbital, hexethal, hexobarbital, mephobarbital, metharbital, methohexital, methylphenobarbital, narcobarbital, nealbarbital, pentobarbital, phenobarbital, phetharbital, prazitone, probarbital, propallylonal, proxibarbal, roxibarbital, reposal, secbutabarbital, secobarbital, sigmodal, spirobarbital, talbutal, thialbarbital, thiamylal, thiobarbital, thiobutabarbital, thiopental, valofane, vinbarbital, and vinylbital.

44. The method as recited in claim 31 wherein said benzodiazepine is selected from the group consisting of alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

45. The method as recited in claim 31 wherein said amphetamine-like stimulant is selected from the group consisting of 4-bromomethcathinone, 4-fluoroamphetamine, 4-fluoromethamphetamine, 4-fluoromethcathinone, 4-methylmethcathinone, aletamine, amfepentorex, amphechloral, racemic amphetamine salts (dextroamphetamine, Adderall), amphetaminil, benzphetamine, bupropion, cathinone, chlorphentermine, clenbuterol, clobenzorex, clortermine, diethylpropion, dimethoxyamphetamine, dimethylamphetamine, dimethylcathinone, ephedrine, epinephrine, ethcathinone, ethylamphetamine, fenethylline, fenfluramine, fenproporex, fludorex, furfenorex, levomethamphetamine, misdexamfetamine, MDMA, mefenorex, methamphetamine, methcathinone, methoxyphedrine, methylone, octopamine, ortetamine, parahydroxyamphetamine, PCA, PIA, PMA, PMEA, PMMA, PPAP, phendimetrazine, phenmetrazine, phentermine, phenylephrine, phenylpropanolamine, propylamphetamine, pseudoephedrine, selegiline, synephrine, tiflorex, and xylopropamine.

46. The method as recited in claim 29, further resulting in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

47. The method as recited in claim 29, further resulting in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

48. The method as recited in claim 29, wherein the method affects a decreased metabolism of the compound per dosage unit thereof by at least one polymorphically-expressed cytochrome P₄₅₀ isoform in the subject, as compared to the corresponding non-isotopically enriched compound.

49. The method as recited in claim 48, wherein the cytochrome P₄₅₀ isoform is selected from the group consisting of CYP2C8, CYP2C9, CYP2C19, and CYP2D6.

50. The method as recited claim 29, wherein said compound is characterized by decreased inhibition of at least one cytochrome P₄₅₀ or monoamine oxidase isoform in said subject per dosage unit thereof as compared to the non-isotopically enriched compound.

51. The method as recited in claim 50, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

52. The method as recited in claim 29, wherein the method reduces a deleterious change in a diagnostic hepatobiliary function endpoint, as compared to the corresponding non-isotopically enriched compound.

53. The method as recited in claim 52, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

54. A compound for use as a medicament, having structural Formula I: 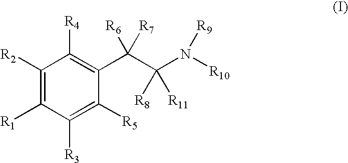
or a pharmaceutically acceptable salt thereof, wherein:
R₁ and R₂ are independently selected from the group consisting of hydrogen, deuterium, —OH, and —OD, wherein at least one of R₁ or R₂ is hydrogen or deuterium;
R₃-R₁₀ are independently selected from the group consisting of hydrogen and deuterium;
R₁₁ is selected from the group consisting of hydrogen, deuterium, CO₂H, —CO₂D, and —CO₂R₁₂, wherein R₁₂ is an alkyl, or deuterated alkyl; and
at least one of R₁-R₁₂ is deuterium or contains deuterium.

55. A compound for use in manufacturing a medicament for the prevention or treatment of a disorder ameliorated by administering a modulator of hormone levels in a subject or a modulator of pigment levels in a subject, having structural Formula I: 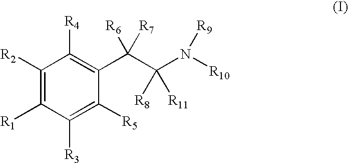
or a pharmaceutically acceptable salt thereof, wherein:
R₁ and R₂ are independently selected from the group consisting of hydrogen, deuterium, —OH, and —OD, wherein at least one of R₁ or R₂ is hydrogen or deuterium;
R₃-R₁₀ are independently selected from the group consisting of hydrogen and deuterium;
R₁₁ is selected from the group consisting of hydrogen, deuterium, CO₂H, —CO₂D, and —CO₂R₁₂, wherein R₁₂ is an alkyl, or deuterated alkyl; and
at least one of R₁-R₁₂ is deuterium or contains deuterium.
